SEQUENCE LISTING (306 sequences)

SEQ ID NO: 1 - DNA, 2202 bp, Artificial Sequence
  synthesized
atgacggtgaaaactaaggctgctaagggcaccctcacttactccaggatgaggaaatcagatccatttctggagttcttccgccagggtaatgggaaatggcacctggtgtacagatctgaggtcatcatggatctggaaagccagaacgcatgtgctgagattgttggtgtccccacccaccccacagctctgaatcgtgtcgtggaggacttcaagaataagtacgaagatgaaattcaccaccgcacagctgctgagaatgagtttgtggtgctgggtgactactcacctttaaggattaaacacagaactgaagatgaaatcacgtaccagtgtagaaatggtttttatgtgccctcccagagaccaacgttcaagcagttggtagatgacttggatcgaattctcactctcacaaccaatgagtgcattgagtgccacctgtcagcagctggccaagcccaagaagaatgtgtggacaagtgcaaactagctggtgcgatgacgacagatttggaagggagcgacatgttggtacaaaaggctgaccggcgggagttcattgacctgttgaagaatggacagtggacactgattggacggcagagccctgagctgccacaggctttctaccctgtggggcatccagttgggggcatcgccagagagtggtttttcctcctgtctgatgaggtgctcaaccctatgtattgtttatttgaatataagcccaacaagcttcgaaatggccacaaggggctttggatcttctgcagtgaagatgagcagagccgcacctgcccggccgagtacatcagtgctgagaggatcgtgtgtaagatggaggagtcgctggtgcccagcccgccgccgggggcaaaggctagactggagctattgggtagacagttggtagcatcagcactctttgcacaatggatggatgttaatctgcagggcggaagcttcgacgtggcagacagaatgttgcacagtgtgaagagcacgtgggagtcggcctccagacggacaccactgcagaaggggcagactgcctatcagcacaaggggctgcctcagctcttctgctcgtcatcctgcactcagcaccttagtcttcaagtaccattacgatctcctagttcatcctcttcctcagaagagaacagtagttctgtggtgcgtcgccagctctttcttgacgtgcttgatgaaaccaaagcattactcctagttccagcctcagtgaacatgcagtacgcactagcagtttccaagcagcggggcccaaaacgctccctagacccgctgggaagtgaggaggagggacgatccactgttcagtttcatatattaggcatgacatgtaagatttgtgaatcctataatactgctcaagctgatggagggtcctcgctgggtttcaagcggagctacaaggaacacatcccttacacacacatgaacggaggcaagaagaggccgctgatgttggcagcattgaatgggctcttggtggctcgagtctcagggcgggaagagaatcgagttgagctgctggagatctgcgagctcagccaggagaagaggagctctgtgtttgaggacagtaacgtgtacatgttgccccagtccccgccggctccccctgtcttcctcagcacctcctgtttcgacagccaagaccactcagccttcgagaaatccacagagagattgcctggtgagcctagaggtaaagagcagattgtccctccaggaaggcagcgagccaggatggagatgggcaccgagatcagccgcaagatccggagtgccattatgatatcccatattatccgagagattcgccagtttgaacaaactgcctacaaaatagagcaccaagcaaaggtagccagcagtgggacttacaacttggactttgacaacatggagcttgtggatacctttcagaccttggagcctcgtagagaggttctgccaggagccaaatgtgattataccttacaggtgcagctaaggttctgtctctgtgagaccagcctcaccaaccagttctatctggctgctccccgaggtgtctgctacggggctgaccatgacctgggccgcctgcactgtccggacatcatcatgaacccacacggcttcccattacgaatgacggtggggaagctcattgagctgctggcttag

SEQ ID NO: 2 - DNA, 2238 bp, Artificial Sequence
  synthesized
atggatgagaaatgcaaacaggttctcaatgtttcccagatggtatatggccctgaaggatcagatctggttttaatgcagttctatctggctgctccccgaggtgcctgctacagggctgaccatgacctgggccgcctgcacccttgtgtgatggtgattttactttgtaacctgaaacctgcaaagataaggggagtattatctcaagcaatggtcatgtgtgctgacagcaacctcactacctacagtcgcccagggcaggctactccttacgccaccactcagctcatccagtcaaactggccagtgttccgggagcagctctgtcgtcacacatgcctcttctacatggttcgggcacaggcctggagccaggaggcctcagccctcggtctaatcctggctgactcatatgaggcctcaggcaagggcaatctggccaggctcattgaagtgaatataaccacaaacctggacttcttcctatgtgtggctcaagttcaactcttacatcagttaatagtaaaagagctggagaaatacaagaacattgatgaagatcagcttcttggcaaactctcagaagaggaactgaaacaggggcccagtgctttcagcatccctctccctatccggcagaagctctgtagcagcctggatgccccccagacgagaagccaaagggtctgcaggcctcgctctatatggagagccaagtttaggttcaatttcttttagtttgctgccaagaaacctccttgtgtagcatatgataaaaagttgcagttctattccaagatagcttatgaggttggcggggcgggtaagatggcggccccgcggcgagggaaaggatcctccacagtgctctcttcagttccccttcaagttcccccccagtattttcagcacctggatcataccgacaaacgtgtggatgcttatgaccgccctgagctatcccggtacaataagatccaggccgactaccacaatctccttggagtcacagcagagctggtggattctctagaggccccttctcttccagacagtaacactattactcaggacttcaaaaccataccttctcagaatagccagatagtaaccgaaagtcccaaacagggcagcaataaaatcaagctccagagtcgcctttcagggggtgtacaccgccttgagtcacagcaagcattcaactatctcaaccaaggggtggttcagtctataacctggaagcagatgaagccacacatacaggagctttctagcctacgttactcttcaccacaagctcctgtcaaggtggaggaaacaagaaaagacttcaggtatccgcggacttctctgcacagcatccctgtgacagtgggggtgaagccggtgctgccaagagccatgcccagttccggtgggctctgtgaaatacctggagtgctcagccctcacccagcggggcctgaagacagtgtttgaaaagtaccatcagaattgatttccctctttaataaaaaatgggatagagagattcttcttaatatccttaccctaggggagatcccagatctctactctgatgatgaagttcaaaacatcataagcaatgtgaggaatgaagtcaagagcgtgaagtctaccaagctcgtgctcaagggaaccaaggcgaagagtaagaagaaaaagagcaaagataagaaaagaaccgagcctgggaggccggccggtgccagcacctttgggcttctgagacggcggcagcagcggcattcagggactccgggcaagggggcgtcgctggatgcaggctcggggaagcccccgatggactaccacgaggatgacaagcgcttcgctagtagtgatcgggccgctgtgtgtggtgagtgtgacaaaagcttcaggcagatgtcagatctggtgaaacaccacggcctggactacctccacaccaagtgcaagatcctccacacggacatcaagcccgagaacatcttgctgtgtccaagcttaatggaacccccctctcccagggaagacaggagattgctttatctgggcttccagctgatgctatttattcttctcttctgctgggcctttgaacaagaagagaaacctaccttcaccaagctcatggacatgctggagaaactcgggtgccagtgccgcggccgcggccgccgccgacgccgccgccgccgccgccgtcgcctgggcggctgcgcctactag

SEQ ID NO: 3 - DNA, 2238 bp, Artificial Sequence
  synthesized
atggctggacccactaatgccatcttcaaagcctttgctgtgatcattgacaaactggaagaggacataagcagctcttggcagcaacagtcttacctggactctggaatccatcctggtgccactaccacagctccttctctgagtggtaaactgctgctgaacctggcagaagccctggcaattttgcgctgtgtggctacgcccctgctcctcgccctattctgcgcacctccaattgcaactggagtcagcagtagtgccactggaccaagcactgctaattacaattcgttctcaagtctgactgcgactcatgaagataaagagaggctggacagctgtacagcatttgctctgatgtatgagggccgccgtgcggtgctggaatatctgacggccgagatcttagagccagctggcaacgcggctcgcgacaataagaagacccgcgtggcatttaataaggaacttgatcctatacagaaaccctttgtggacaagattagagaatacaaatctaagcgacaagggatagaattctggtccaatgtctgtgatgagaaaatggatttggccattgaagcttcagaggcagcagaatcaagcttcctggcaggcgaagcatcctggctgaatgaactggatgggcacaatgacacgggcttggtctttgccagtaagtcccctgttgggtcacctctagggagcgcctttgcagaggcccccagcgagatgccaggggatgaggtgaagaagtacagccgcaatgctctcctcttcaacctgtgcttcgtgtgtgatgcccaggccaagacctgcgccctcaacacgcactatgtggcaccccgccgcctgctgctgatgggcacaccgctgcagaacaagcttcccgagctctggtctcccaacatggggatgggcactagtggaccaaatagggtcctacgcagtcaacaggtacaccctaatgggcagattgtccagcttctacctttgatcagcttcgaggctctaatacccagcccaacttacagcctgtcatgtttcggaacccagggtctgcttaatctggactgcagagagtataacgcagacaaggtcatcgtggacagtggcaccacgctgctgcgcctgcccacctggagcgtcgccttctccccagatggctcctggttagcttggtctcaaggacactgcatcgtcaaactgatcatccccttgaactaccacatagttgaggtgatctttgtagagctgtttcaacttccagcaccccctcacattgatttgctcggcgcatttccttcacctagtggtcctgcctctgtagtcttgagtggccggactcaagctcagaccaggcaagctagtgttctaattgacaggcctgccccacacttcgagcgtacatcctcactgcggcatcgacaccccagtctggctttccgaaaactgtctgagggctcctctgaccaggacgggtactttaatgcgccatgggaaaaacgagttgacctctcttggcaattaatggaaatagcagaacagcttacaaacgcagagttcctcgtgggggaccttgttgtcacccaggacacttccctaccctggcccagctcctatctcactaagagccgggcctgctacaacctgacagcactggccttgggcccccagagccggctggacaccttcgattacgactggactaagaatgccgtgctcacgtaccccagccctgccacgctggggcacctccaggcggaggctgagaagctggagatctgcatggagctcatggacacatccttggacaagctctaccggaaggtgctggataaaaacatgacaattccaatagctgccaattttcccttcttggattccttctgtgctctggacaaagatcaggatggcctaattagtaaagatagcgagagcttcggggtccccaaggggaggagccagctcaaagtgtcgaaatctccgtgtgaaaagctgatcagcactagagtgaagcaaattgccaaattgtggagaaaatcaagctcacaagaaagagcaccatatgtgcaaaaagccggcctcatcgtggttagcaaaacaggagactgggtgggaaagtggacctccacctccatgccctgggcagccgcccaagtggaacttctacggcagatgaacgaacaacatgaaaaggtttatgaacaattagacgtcacagcaagggaatag

SEQ ID NO: 4 - DNA, 2229 bp, Artificial Sequence
  synthesized
atgcgctggtggattgtggggtccgcctggagtggggcccggatgatcgacaacagtcaccatacgcacctgcagaagtccagcttcacccctaagcagcgcagcaaggctcgtacctgttcagaaggccgggagtgtgtcaactgtggggccctgtttctgctgtgtgacaaggaggctaagggcttcgtcaccaagcacgacctgcagggtctccagagcgacctgggtggccgctcgcccaccgtgcgtgtgtcagtggtgaccaacacgccctcgggcccggtggaggcctttgactttcagcagcaggtggtgcagctagaagggctggagaatgtcactgcccggaaccttctgggcaaactcatcaacatcgctgcgcaggtcggagcggtgcgcgtagtacgggcggttggcggcgcaggaggagccggacaaagaggggaaggagaaacctcatgctggggtctccccgcggggagtatggccaagccttggctgctagttctctacttcgctctgattgtggttccgatggcccaggtcagcatcaacaatgactacagcgagttggacttgagcacggatgccggggagcgggctcggctgcccgcgcgccgccgccgcctctactggggcttcttcttgggccgcggccgcgtcaagccgggggggcgctggcgcctggccaaacctaagagcgggacgtttcaggccctgaaggtggcctccagtgtggcatctgcctacgatgagatgagtgactgcaccggcctcccactgtccatgctgaccagcccagccacacccccggcccggggccagtaccaaatcgctgccgcgttaggctatagggagcgactagacatggtgcccgataccctgcagaaacaggcggaccactgtaacaccagggaagctctggctgagagcgaccgtgcagctaagggtctgggggctggagttgaggaggaaggagatgatgaaggaggggtctctacagagacagaaaagccaaaactaaagtttaaggaaaaaaataaaaatagtgatggaggaattctgcaagcaactgggttcccatggcatgtggtggtcttagaggaggtgttcagcctgccaccgtcggtgcttaagcaagccctgaaaaagcccatcaagggcaaacagcccccccgaaaaaaagctcaaggaaaaacgcaacagaatgccgccctgggcaagcaggcgagcttctcgggcagcctggaggtcatcgaggtgtccgggctggagctggaggagctggtgccgcgcctgttcctggtggcgtccaaaagacacggggaccttctggaggtaatggcccaggcgtctcgctcaggtagcctgcttccactcgttatcgtgcccccgctgagggcgcaacccgaagaagctgtggtcatggtagaggttaaccagagcgcaaagaaacaagtcatcttgtcctcagggacagaattctccattctggtgcagtgccactacaggctccaggatgccaaagctcagaaggtgtggtgccggttcttgccggaggagctgtcgagtcagctcttctactacggaggggaaccgcagagccagaagagcacggagcagcaactcgcagccgacaccaccctctacataccgagaacggcggacaaaactctttttccaggtcatctgcacgacgcagcgctcaacctcactgacacccaagtcaagaccttgtaccagaaccgcaggaccaagtggaagcggcagacaatgagtaaatatccatctggtgagagagtacgttatcaatgtaggagcccttatgaaatgtttggggatgaagaacttaatgtccttgacaacaaaaagaggacagctctgacaaaggccgtacaatgccaggaagatgaatgtgcgttagcaaaagcattttggagaatggataggcttttaggtcatggaagaaaagaagtgtccagggttcaatggagtggccgaccactgtgcaagtttgctcatgtgcctgaacaaagggatgaaaaggtttgcatggatgtgtttaagaaatatctcgcccccaaagggcaacccggacccgctgccactaataggaaccctagaggctccagcagcagacagagcagctttgtgaaagcatcaaagcgctttgaagagtccaaagacattactccaggcccagcaacatatgagatatcccagtag

SEQ ID NO: 5 - DNA, 2433 bp, Artificial Sequence
  synthesized
atggcacagcctgctcaacctgctgatgaacctgcagaaagggctgatgaaccaatggaacataagaagatggatctcattgacctagaggatgagaccgttgatgccgaggtcatgaactctctagcagttactatgggcataaccatccctggtatctcttcagatgtgccagacagtgcccacaaaactactgcctcagaggcaaagtcagccagacctcatccattccaaaatatgcccggtgctacccgcccagctgctcctagaccaccatttagtactatggtcacccacgctcctcagcccctgcttcccggttccgacaggcccctgccctgtgggccccaggaggccgcatgcggcctgccatcgctgccaccaccacctgcggcccctacctcagggccgcccctgagcgccacgcagatcaaacagtacaagcagtcacagcacatgacggaggttgtgaggcactgcccccaccatgagcgctgctcagatagcgatggtcttgagaaacttcgccagcgaatacatgataaagctgtgctctggagcgggctacgtgatatgaagagctgtcagtttgtagccgtcagaggagatactggagaaaagctgacagttgctgaaaatgagtttctgctgcacccgagtgatcatgaaaatattcagacattttcagatgaatttgccagaagggctaatggaaattatgctaaaaataaagcagttggagtgatgaagtcgagccatgtatttacaattgagccaatgatttgtgaaggcggcgagccggtggagttcagatgcatcctggaggctcataatgttcccgaccgttactttgctgtctcctgggccctcaatggatgtgaaaatggtcgctgtgtgagggtcaaggaaggttacacctgcgattgctttgatgggtatcacaaaactgaaacaatgaatgtagttatggagaccaatagaatgctaagagaagagaaggagagactagaacaggatcatggagacaaagacaaaatggttgctgttgcctcacagagtagcatcatcttgtggagtgttcaggatgggggagacaagaacaaggccaacagagagtcactctatgacgtttgggagtactatctcccctacctctactcatgcatcggcatcctgcttccccagttcctgggggtgctgctgatgctgctgtacatcacccgggtggaggacatcatcatgatgaacaacttcctgggcagtgagcccatcctgatgctcacactgggatcccagcctgtcctcaagaccgacaacagaaatgctactggtcctcgtcctgctttatttgtggctgaggtttcatttgagttactggtgaagcggcaaatccttcacgtggagaagttcatcagcgtgcacgagaacggccagagcagtgctgatggtgccagtgaacgtgctgttctgctccagggcaggggcctgcaagaggaagaggagttctgcatggccccgcgcagctctcagagctcagaagaggtcatccttggcatggatgatacacaggtgcgaagttctgctactgatatattctcatacttggttgaatataatgacagtaagctcaaggacatgcgagatgaggtcctgcaacatgagctgatggtggctgaccggcgccagaagcgatgcgagctgcccagcaccatccccatcaagcagaacacccgccgcagcagctggagctctgacgaagggatcgggtgcacagaagctgtttttttattattgggaatgctccacagcatgctgacttcaccttggcaatcagaagaagagctcaaacgcagcaaagcctccatctcctcctcctcctcctcagccccagcgatgccagcacttcctcattccccaccagctctgatggcctctctcccttctcggagaccttcatccctgactccagcaagggccttggccccagggcacccgcatccccggcaccagcccaggcccagacccccacccctgctccagccccagcctcctcccaagcaccccttcggccagaggatgacattctgtaccagtggcggcagcggcggaagcttgaacaggctcagggaagcaagggtgacagagcttgggtgccgcctccaagttgtggttcctggtataaatttacccgtcaatcatctgacctatttcttcacggcagttctcattagtggtcacatcaaggccaagcacaggaatgagaggccacacgcatgtgagttctgcagccacgccttcacccaaaaggccgacctagtggaagggttacatgatggacacttctacaccctgaagcgaatcctgtgtcacgagcagcaggaccggatgactggcatcttccattttgccattatcttcatgttaatactttaa

SEQ ID NO: 6 - DNA, 2358 bp, Artificial Sequence
  synthesized
atgaggctccatccagtcccagtcccagcagattggatgaaacgcatggaggtgaatgagacccagtacaatgagatgacttccttctgcgtctgcacaggccctgggcaccagcaggctatgctttcacccacctcagcctgcaggagtttcttgctgccctgcaccagaggtggccgggggcgcccaagggggcagcctcctatgcccccaaagccttccatcaggccccggccagggagcatgtccctcgagagaccccccagggtgcctccgagatctgcctcacagaggcctccgaccagggagaccttccatccctttgctgatgatgtggaggacacctatgacgacgagactgaggactcaagcctgcaggagaaattcttcacgttccctaaggccccttctaagctgacagtggggggggccatggggccaaagtcaggaaaaggaaaagggagacaagaattcagccaccccgaaacagaagccattgcctcccttgtttaccttgggtccacctccaccaaaacccaacagaccaccaaatgtaccaagaacaaccagttccaggccctgctgcagtatgtggaccccgtgagcgcccagcacgccaagctgtcgctgagacctgccagcccttcagcaccactgcaggataacggaactcaaggcttaattaacggggcactaaacaaaacagaaaggatccgagatctaagtggcaatctttgggagcattccagtggggatggagaagaactagaaagacttaccgaagttagcatgatagtaaattcacataagccccaaaagaatttggattctatacaagttaccaaagatctcacatccttaaagttttttcagaaaaaggaaactaagagaggtttggatttcacagattctcaagaaaatgaagaaaaagcttcctgtggcacagagggatttcggaatgccaaggagggcacaggcatcgcagcacagacagcaggcatagccctgtggccttcagacagcgccgcaccgcggctgggctcctcggagatcatgcatgccccgcggtccagggacagggccactgaattttttgaagatgacgccacattaaccccttcattttgtttggaagatggaccaaagcgatacactaacagtgaatgtgcaagtaaagatgttctggtgaagaacctcagggctattgatgtgaaacttaactctgataatatggagcaagagaggaatgctcgctaccgaacccccagaatcttggagcccacagccttccaggaacccccacccccccctttgtgggaaaagcctcccttgggaagcagtagttgtatgctcagtcctcccctgggaagaacaacaacacctaccaccacccggggtctctgggcaataagtgagagagagcgatctatgaaagcaatactatcatttgcaaaaataaagcagttaaacatttcacacttactagtacctccgggattagccaaaccagctcatctttatgacaaagggtgtgtagaagagaggatatgtcacctccaacgtgaaattgcgtggcttgtacagcaactagatgacgttcatcagtttggttcagtgaccaacataccagcaaaggatgcaccaccagtggagaaaatagacaccatgctggtcttggaacttgggcttgccttcaacttgaatccagctctgaccataattggcaagtatttctacaagaggcgaccattggccagccagcttccccattgcccctctgtatctcccagtacccagccttggacacaccctggccagagccagctgtttgacttcacttgtgatgacggaaatgatgacaatagttactccccactttctcgctgtcctactgaatgtacttgcaaacacgatgaaaacctgtatggtcccattgtgaaacgtatttcattttttgatttcttcaagcataagtatcaacacctcaaagctcttaaatgcttatctctggtacccgcagtcatgggacaactcaaattcaatacgtcggaggaaggagccagcatattccagtccaaagagtcctcgtcagagcttagctgtgacatctctgtggatgataagtacatatacatgagacggcccaatccagttagtccttatccaatctcttcacacacttcagatatctatggaagcaccagcttcaaccatcacccctacccccacaaccactacatggcggatttgcaccctgctgcaggccaccagatgaacgggaagtggggtggctgtagcgaggacatcgagtttggtgagatggtgtctcgggagttcgccgacgcccgggagaactga

SEQ ID NO: 7 - protein, 18 aa, Artificial Sequence
  synthesized
MTVKTKAAKGTLTYSRMR

SEQ ID NO: 8 - protein, 25 aa, Artificial Sequence
  synthesized
KSDPFLEFFRQGNGKWHLVYRSEVI

SEQ ID NO: 9 - protein, 25 aa, Artificial Sequence
  synthesized
MDLESQNACAEIVGVPTHPTALNRV

SEQ ID NO: 10 - protein, 25 aa, Artificial Sequence
  synthesized
VEDFKNKYEDEIHHRTAAENEFVVL

SEQ ID NO: 11 - protein, 25 aa, Artificial Sequence
  synthesized
GDYSPLRIKHRTEDEITYQCRNGFY

SEQ ID NO: 12 - protein, 25 aa, Artificial Sequence
  synthesized
VPSQRPTFKQLVDDLDRILTLTTNE

SEQ ID NO: 13 - protein, 25 aa, Artificial Sequence
  synthesized
CIECHLSAAGQAQEECVDKCKLAGA

SEQ ID NO: 14 - protein, 25 aa, Artificial Sequence
  synthesized
MTTDLEGSDMLVQKADRREFIDLLK

SEQ ID NO: 15 - protein, 25 aa, Artificial Sequence
  synthesized
NGQWTLIGRQSPELPQAFYPVGHPV

SEQ ID NO: 16 - protein, 25 aa, Artificial Sequence
  synthesized
GGIAREWFFLLSDEVLNPMYCLFEY

SEQ ID NO: 17 - protein, 25 aa, Artificial Sequence
  synthesized
KPNKLRNGHKGLWIFCSEDEQSRTC

SEQ ID NO: 18 - protein, 25 aa, Artificial Sequence
  synthesized
PAEYISAERIVCKMEESLVPSPPPG

SEQ ID NO: 19 - protein, 25 aa, Artificial Sequence
  synthesized
AKARLELLGRQLVASALFAQWMDVN

SEQ ID NO: 20 - protein, 25 aa, Artificial Sequence
  synthesized
LQGGSFDVADRMLHSVKSTWESASR

SEQ ID NO: 21 - protein, 25 aa, Artificial Sequence
  synthesized
RTPLQKGQTAYQHKGLPQLFCSSSC

SEQ ID NO: 22 - protein, 25 aa, Artificial Sequence
  synthesized
TQHLSLQVPLRSPSSSSSSEENSSS

SEQ ID NO: 23 - protein, 25 aa, Artificial Sequence
  synthesized
VVRRQLFLDVLDETKALLLVPASVN

SEQ ID NO: 24 - protein, 25 aa, Artificial Sequence
  synthesized
MQYALAVSKQRGPKRSLDPLGSEEE

SEQ ID NO: 25 - protein, 25 aa, Artificial Sequence
  synthesized
GRSTVQFHILGMTCKICESYNTAQA

SEQ ID NO: 26 - protein, 25 aa, Artificial Sequence
  synthesized
DGGSSLGFKRSYKEHIPYTHMNGGK

SEQ ID NO: 27 - protein, 25 aa, Artificial Sequence
  synthesized
KRPLMLAALNGLLVARVSGREENRV

SEQ ID NO: 28 - protein, 25 aa, Artificial Sequence
  synthesized
ELLEICELSQEKRSSVFEDSNVYML

SEQ ID NO: 29 - protein, 25 aa, Artificial Sequence
  synthesized
PQSPPAPPVFLSTSCFDSQDHSAFE

SEQ ID NO: 30 - protein, 25 aa, Artificial Sequence
  synthesized
KSTERLPGEPRGKEQIVPPGRQRAR

SEQ ID NO: 31 - protein, 15 aa, Artificial Sequence
  synthesized
MEMGTEISRKIRSAI

SEQ ID NO: 32 - protein, 25 aa, Artificial Sequence
  synthesized
MISHIIREIRQFEQTAYKIEHQAKV

SEQ ID NO: 33 - protein, 25 aa, Artificial Sequence
  synthesized
ASSGTYNLDFDNMELVDTFQTLEPR

SEQ ID NO: 34 - protein, 25 aa, Artificial Sequence
  synthesized
REVLPGAKCDYTLQVQLRFCLCETS

SEQ ID NO: 35 - protein, 25 aa, Artificial Sequence
  synthesized
LTNQFYLAAPRGVCYGADHDLGRLH

SEQ ID NO: 36 - protein, 25 aa, Artificial Sequence
  synthesized
CPDIIMNPHGFPLRMTVGKLIELLA

SEQ ID NO: 37 - protein, 25 aa, Artificial Sequence
  synthesized
DEKCKQVLNVSQMVYGPEGSDLVLM

SEQ ID NO: 38 - protein, 25 aa, Artificial Sequence
  synthesized
QFYLAAPRGACYRADHDLGRLHPCV

SEQ ID NO: 39 - protein, 25 aa, Artificial Sequence
  synthesized
MVILLCNLKPAKIRGVLSQAMVMCA

SEQ ID NO: 40 - protein, 25 aa, Artificial Sequence
  synthesized
DSNLTTYSRPGQATPYATTQLIQSN

SEQ ID NO: 41 - protein, 25 aa, Artificial Sequence
  synthesized
WPVFREQLCRHTCLFYMVRAQAWSQ

SEQ ID NO: 42 - protein, 25 aa, Artificial Sequence
  synthesized
EASALGLILADSYEASGKGNLARLI

SEQ ID NO: 43 - protein, 25 aa, Artificial Sequence
  synthesized
EVNITTNLDFFLCVAQVQLLHQLIV

SEQ ID NO: 44 - protein, 25 aa, Artificial Sequence
  synthesized
KELEKYKNIDEDQLLGKLSEEELKQ

SEQ ID NO: 45 - protein, 25 aa, Artificial Sequence
  synthesized
GPSAFSIPLPIRQKLCSSLDAPQTR

SEQ ID NO: 46 - protein, 20 aa, Artificial Sequence
  synthesized
SQRVCRPRSIWRAKFRFNFF

SEQ ID NO: 47 - protein, 25 aa, Artificial Sequence
  synthesized
FAAKKPPCVAYDKKLQFYSKIAYEV

SEQ ID NO: 48 - protein, 25 aa, Artificial Sequence
  synthesized
GGAGKMAAPRRGKGSSTVLSSVPLQ

SEQ ID NO: 49 - protein, 25 aa, Artificial Sequence
  synthesized
VPPQYFQHLDHTDKRVDAYDRPELS

SEQ ID NO: 50 - protein, 25 aa, Artificial Sequence
  synthesized
RYNKIQADYHNLLGVTAELVDSLEA

SEQ ID NO: 51 - protein, 25 aa, Artificial Sequence
  synthesized
PSLPDSNTITQDFKTIPSQNSQIVT

SEQ ID NO: 52 - protein, 25 aa, Artificial Sequence
  synthesized
ESPKQGSNKIKLQSRLSGGVHRLES

SEQ ID NO: 53 - protein, 25 aa, Artificial Sequence
  synthesized
QQAFNYLNQGVVQSITWKQMKPHIQ

SEQ ID NO: 54 - protein, 25 aa, Artificial Sequence
  synthesized
ELSSLRYSSPQAPVKVEETRKDFRY

SEQ ID NO: 55 - protein, 25 aa, Artificial Sequence
  synthesized
PRTSLHSIPVTVGVKPVLPRAMPSS

SEQ ID NO: 56 - protein, 21 aa, Artificial Sequence
  synthesized
GGLCEIPGVLSPHPAGPEDSV

SEQ ID NO: 57 - protein, 25 aa, Artificial Sequence
  synthesized
KVPSELISLFNKKWDREILLNILTL

SEQ ID NO: 58 - protein, 25 aa, Artificial Sequence
  synthesized
GEIPDLYSDDEVQNIISNVRNEVKS

SEQ ID NO: 59 - protein, 25 aa, Artificial Sequence
  synthesized
VKSTKLVLKGTKAKSKKKKSKDKKR

SEQ ID NO: 60 - protein, 25 aa, Artificial Sequence
  synthesized
TEPGRPAGASTFGLLRRRQQRHSGT

SEQ ID NO: 61 - protein, 25 aa, Artificial Sequence
  synthesized
PGKGASLDAGSGKPPMDYHEDDKRF

SEQ ID NO: 62 - protein, 25 aa, Artificial Sequence
  synthesized
ASSDRAAVCGECDKSFRQMSDLVKH

SEQ ID NO: 63 - protein, 25 aa, Artificial Sequence
  synthesized
HGLDYLHTKCKILHTDIKPENILLC

SEQ ID NO: 64 - protein, 25 aa, Artificial Sequence
  synthesized
PSLMEPPSPREDRRLLYLGFQLMLF

SEQ ID NO: 65 - protein, 25 aa, Artificial Sequence
  synthesized
ILLFCWAFEQEEKPTFTKLMDMLEK

SEQ ID NO: 66 - protein, 26 aa, Artificial Sequence
  synthesized
LGCQCRGRGRRRRRRRRRRRLGGCAY

SEQ ID NO: 67 - protein, 25 aa, Artificial Sequence
  synthesized
AGPTNAIFKAFAVIIDKLEEDISSS

SEQ ID NO: 68 - protein, 25 aa, Artificial Sequence
  synthesized
WQQQSYLDSGIHPGATTTAPSLSGK

SEQ ID NO: 69 - protein, 25 aa, Artificial Sequence
  synthesized
LLLNLAEALAILRCVATPLLLALFC

SEQ ID NO: 70 - protein, 25 aa, Artificial Sequence
  synthesized
APPIATGVSSSATGPSTANYNSFSS

SEQ ID NO: 71 - protein, 25 aa, Artificial Sequence
  synthesized
LTATHEDKERLDSCTAFALMYEGRR

SEQ ID NO: 72 - protein, 25 aa, Artificial Sequence
  synthesized
AVLEYLTAEILEPAGNAARDNKKTR

SEQ ID NO: 73 - protein, 25 aa, Artificial Sequence
  synthesized
VAFNKELDPIQKPFVDKIREYKSKR

SEQ ID NO: 74 - protein, 25 aa, Artificial Sequence
  synthesized
QGIEFWSNVCDEKMDLAIEASEAAE

SEQ ID NO: 75 - protein, 25 aa, Artificial Sequence
  synthesized
SSFLAGEASWLNELDGHNDTGLVFA

SEQ ID NO: 76 - protein, 25 aa, Artificial Sequence
  synthesized
SKSPVGSPLGSAFAEAPSEMPGDEV

SEQ ID NO: 77 - protein, 25 aa, Artificial Sequence
  synthesized
KKYSRNALLFNLCFVCDAQAKTCAL

SEQ ID NO: 78 - protein, 25 aa, Artificial Sequence
  synthesized
NTHYVAPRRLLLMGTPLQNKLPELW

SEQ ID NO: 79 - protein, 20 aa, Artificial Sequence
  synthesized
SPNMGMGTSGPNRVLRSQQV

SEQ ID NO: 80 - protein, 34 aa, Artificial Sequence
  synthesized
HPNGQIVQLLPLISFEALIPSPTYSLSCFGTQGL

SEQ ID NO: 81 - protein, 25 aa, Artificial Sequence
  synthesized
LNLDCREYNADKVIVDSGTTLLRLP

SEQ ID NO: 82 - protein, 25 aa, Artificial Sequence
  synthesized
TWSVAFSPDGSWLAWSQGHCIVKLI

SEQ ID NO: 83 - protein, 25 aa, Artificial Sequence
  synthesized
IPLNYHIVEVIFVELFQLPAPPHID

SEQ ID NO: 84 - protein, 16 aa, Artificial Sequence
  synthesized
LLGAFPSPSGPASVVL

SEQ ID NO: 85 - protein, 25 aa, Artificial Sequence
  synthesized
SGRTQAQTRQASVLIDRPAPHFERT

SEQ ID NO: 86 - protein, 24 aa, Artificial Sequence
  synthesized
SSLRHRHPSLAFRKLSEGSSDQDG

SEQ ID NO: 87 - protein, 25 aa, Artificial Sequence
  synthesized
YFNAPWEKRVDLSWQLMEIAEQLTN

SEQ ID NO: 88 - protein, 25 aa, Artificial Sequence
  synthesized
AEFLVGDLVVTQDTSLPWPSSYLTK

SEQ ID NO: 89 - protein, 25 aa, Artificial Sequence
  synthesized
SRACYNLTALALGPQSRLDTFDYDW

SEQ ID NO: 90 - protein, 25 aa, Artificial Sequence
  synthesized
TKNAVLTYPSPATLGHLQAEAEKLE

SEQ ID NO: 91 - protein, 25 aa, Artificial Sequence
  synthesized
ICMELMDTSLDKLYRKVLDKNMTIP

SEQ ID NO: 92 - protein, 25 aa, Artificial Sequence
  synthesized
IAANFPFLDSFCALDKDQDGLISKD

SEQ ID NO: 93 - protein, 25 aa, Artificial Sequence
  synthesized
SESFGVPKGRSQLKVSKSPCEKLIS

SEQ ID NO: 94 - protein, 25 aa, Artificial Sequence
  synthesized
TRVKQIAKLWRKSSSQERAPYVQKA

SEQ ID NO: 95 - protein, 25 aa, Artificial Sequence
  synthesized
GLIVVSKTGDWVGKWTSTSMPWAAA

SEQ ID NO: 96 - protein, 25 aa, Artificial Sequence
  synthesized
QVELLRQMNEQHEKVYEQLDVTARE

SEQ ID NO: 97 - protein, 25 aa, Artificial Sequence
  synthesized
RWWIVGSAWSGARMIDNSHHTHLQK

SEQ ID NO: 98 - protein, 25 aa, Artificial Sequence
  synthesized
SSFTPKQRSKARTCSEGRECVNCGA

SEQ ID NO: 99 - protein, 25 aa, Artificial Sequence
  synthesized
LFLLCDKEAKGFVTKHDLQGLQSDL

SEQ ID NO: 100 - protein, 25 aa, Artificial Sequence
  synthesized
GGRSPTVRVSVVTNTPSGPVEAFDF

SEQ ID NO: 101 - protein, 25 aa, Artificial Sequence
  synthesized
QQQVVQLEGLENVTARNLLGKLINI

SEQ ID NO: 102 - protein, 36 aa, Artificial Sequence
  synthesized
AAQVGAVRVVRAVGGAGGAGQRGEGETSCWGLPAGS

SEQ ID NO: 103 - protein, 17 aa, Artificial Sequence
  synthesized
MAKPWLLVLYFALIVVP

SEQ ID NO: 104 - protein, 25 aa, Artificial Sequence
  synthesized
MAQVSINNDYSELDLSTDAGERARL

SEQ ID NO: 105 - protein, 25 aa, Artificial Sequence
  synthesized
PARRRRLYWGFFLGRGRVKPGGRWR

SEQ ID NO: 106 - protein, 25 aa, Artificial Sequence
  synthesized
LAKPKSGTFQALKVASSVASAYDEM

SEQ ID NO: 107 - protein, 25 aa, Artificial Sequence
  synthesized
SDCTGLPLSMLTSPATPPARGQYQI

SEQ ID NO: 108 - protein, 25 aa, Artificial Sequence
  synthesized
AAALGYRERLDMVPDTLQKQADHCN

SEQ ID NO: 109 - protein, 25 aa, Artificial Sequence
  synthesized
TREALAESDRAAKGLGAGVEEEGDD

SEQ ID NO: 110 - protein, 25 aa, Artificial Sequence
  synthesized
EGGVSTETEKPKLKFKEKNKNSDGG

SEQ ID NO: 111 - protein, 25 aa, Artificial Sequence
  synthesized
ILQATGFPWHVVVLEEVFSLPPSVL

SEQ ID NO: 112 - protein, 25 aa, Artificial Sequence
  synthesized
KQALKKPIKGKQPPRKKAQGKTQQN

SEQ ID NO: 113 - protein, 19 aa, Artificial Sequence
  synthesized
AALGKQASFSGSLEVIEVS

SEQ ID NO: 114 - protein, 25 aa, Artificial Sequence
  synthesized
GLELEELVPRLFLVASKRHGDLLEV

SEQ ID NO: 115 - protein, 23 aa, Artificial Sequence
  synthesized
MAQASRSGSLLPLVIVPPLRAQP

SEQ ID NO: 116 - protein, 25 aa, Artificial Sequence
  synthesized
EEAVVMVEVNQSAKKQVILSSGTEF

SEQ ID NO: 117 - protein, 25 aa, Artificial Sequence
  synthesized
SILVQCHYRLQDAKAQKVWCRFLPE

SEQ ID NO: 118 - protein, 25 aa, Artificial Sequence
  synthesized
ELSSQLFYYGGEPQSQKSTEQQLAA

SEQ ID NO: 119 - protein, 21 aa, Artificial Sequence
  synthesized
DTTLYIPRTADKTLFPGHLHD

SEQ ID NO: 120 - protein, 25 aa, Artificial Sequence
  synthesized
AALNLTDTQVKTLYQNRRTKWKRQT

SEQ ID NO: 121 - protein, 25 aa, Artificial Sequence
  synthesized
MSKYPSGERVRYQCRSPYEMFGDEE

SEQ ID NO: 122 - protein, 25 aa, Artificial Sequence
  synthesized
LNVLDNKKRTALTKAVQCQEDECAL

SEQ ID NO: 123 - protein, 25 aa, Artificial Sequence
  synthesized
AKAFWRMDRLLGHGRKEVSRVQWSG

SEQ ID NO: 124 - protein, 25 aa, Artificial Sequence
  synthesized
RPLCKFAHVPEQRDEKVCMDVFKKY

SEQ ID NO: 125 - protein, 25 aa, Artificial Sequence
  synthesized
LAPKGQPGPAATNRNPRGSSSRQSS

SEQ ID NO: 126 - protein, 25 aa, Artificial Sequence
  synthesized
FVKASKRFEESKDITPGPATYEISQ

SEQ ID NO: 127 - protein, 20 aa, Artificial Sequence
  synthesized
AQPAQPADEPAERADEPMEH

SEQ ID NO: 128 - protein, 25 aa, Artificial Sequence
  synthesized
KKMDLIDLEDETVDAEVMNSLAVTM

SEQ ID NO: 129 - protein, 25 aa, Artificial Sequence
  synthesized
GITIPGISSDVPDSAHKTTASEAKS

SEQ ID NO: 130 - protein, 25 aa, Artificial Sequence
  synthesized
ARPHPFQNMPGATRPAAPRPPFSTM

SEQ ID NO: 131 - protein, 25 aa, Artificial Sequence
  synthesized
VTHAPQPLLPGSDRPLPCGPQEAAC

SEQ ID NO: 132 - protein, 25 aa, Artificial Sequence
  synthesized
GLPSLPPPPAAPTSGPPLSATQIKQ

SEQ ID NO: 133 - protein, 25 aa, Artificial Sequence
  synthesized
YKQSQHMTEVVRHCPHHERCSDSDG

SEQ ID NO: 134 - protein, 18 aa, Artificial Sequence
  synthesized
LEKLRQRIHDKAVLWSGL

SEQ ID NO: 135 - protein, 25 aa, Artificial Sequence
  synthesized
RDMKSCQFVAVRGDTGEKLTVAENE

SEQ ID NO: 136 - protein, 25 aa, Artificial Sequence
  synthesized
FLLHPSDHENIQTFSDEFARRANGN

SEQ ID NO: 137 - protein, 25 aa, Artificial Sequence
  synthesized
YAKNKAVGVMKSSHVFTIEPMICEG

SEQ ID NO: 138 - protein, 25 aa, Artificial Sequence
  synthesized
GEPVEFRCILEAHNVPDRYFAVSWA

SEQ ID NO: 139 - protein, 25 aa, Artificial Sequence
  synthesized
LNGCENGRCVRVKEGYTCDCFDGYH

SEQ ID NO: 140 - protein, 25 aa, Artificial Sequence
  synthesized
KTETMNVVMETNRMLREEKERLEQD

SEQ ID NO: 141 - protein, 25 aa, Artificial Sequence
  synthesized
HGDKDKMVAVASQSSIILWSVQDGG

SEQ ID NO: 142 - protein, 25 aa, Artificial Sequence
  synthesized
DKNKANRESLYDVWEYYLPYLYSCI

SEQ ID NO: 143 - protein, 25 aa, Artificial Sequence
  synthesized
GILLPQFLGVLLMLLYITRVEDIIM

SEQ ID NO: 144 - protein, 25 aa, Artificial Sequence
  synthesized
MNNFLGSEPILMLTLGSQPVLKTDN

SEQ ID NO: 145 - protein, 25 aa, Artificial Sequence
  synthesized
RNATGPRPALFVAEVSFELLVKRQI

SEQ ID NO: 146 - protein, 25 aa, Artificial Sequence
  synthesized
LHVEKFISVHENGQSSADGASERAV

SEQ ID NO: 147 - protein, 25 aa, Artificial Sequence
  synthesized
LLQGRGLQEEEEFCMAPRSSQSSEE

SEQ ID NO: 148 - protein, 25 aa, Artificial Sequence
  synthesized
VILGMDDTQVRSSATDIFSYLVEYN

SEQ ID NO: 149 - protein, 25 aa, Artificial Sequence
  synthesized
DSKLKDMRDEVLQHELMVADRRQKR

SEQ ID NO: 150 - protein, 25 aa, Artificial Sequence
  synthesized
CELPSTIPIKQNTRRSSWSSDEGIG

SEQ ID NO: 151 - protein, 25 aa, Artificial Sequence
  synthesized
CTEAVFLLLGMLHSMLTSPWQSEEE

SEQ ID NO: 152 - protein, 106 aa, Artificial Sequence
  synthesized
LKRSKASISSSSSSAPAMPALPHSPPALMASLPSRRPSSLTPARALAPGHPHPRHQPRPRPPPLLQPQPPPKHPFGQRMTFCTSGGSGGSLNRLREARVTELGCRL

SEQ ID NO: 153 - protein, 25 aa, Artificial Sequence
  synthesized
QVVVPGINLPVNHLTYFFTAVLISG

SEQ ID NO: 154 - protein, 25 aa, Artificial Sequence
  synthesized
HIKAKHRNERPHACEFCSHAFTQKA

SEQ ID NO: 155 - protein, 25 aa, Artificial Sequence
  synthesized
DLVEGLHDGHFYTLKRILCHEQQDR

SEQ ID NO: 156 - protein, 15 aa, Artificial Sequence
  synthesized
MTGIFHFAIIFMLIL

SEQ ID NO: 157 - protein, 25 aa, Artificial Sequence
  synthesized
RLHPVPVPADWMKRMEVNETQYNEM

SEQ ID NO: 158 - protein, 30 aa, Artificial Sequence
  synthesized
TSFCVCTGPGHQQAMLSPTSACRSFLLPCT

SEQ ID NO: 159 - protein, 25 aa, Artificial Sequence
  synthesized
RGGRGRPRGQPPMPPKPSIRPRPGS

SEQ ID NO: 160 - protein, 25 aa, Artificial Sequence
  synthesized
MSLERPPRVPPRSASQRPPTRETFH

SEQ ID NO: 161 - protein, 25 aa, Artificial Sequence
  synthesized
PFADDVEDTYDDETEDSSLQEKFFT

SEQ ID NO: 162 - protein, 54 aa, Artificial Sequence
  synthesized
FPKAPSKLTVGGAMGPKSGKGKGRQEFSHPETEAIASLVYLGSTSTKTQQTTKC

SEQ ID NO: 163 - protein, 25 aa, Artificial Sequence
  synthesized
TKNNQFQALLQYVDPVSAQHAKLSL

SEQ ID NO: 164 - protein, 25 aa, Artificial Sequence
  synthesized
RPASPSAPLQDNGTQGLINGALNKT

SEQ ID NO: 165 - protein, 25 aa, Artificial Sequence
  synthesized
ERIRDLSGNLWEHSSGDGEELERLT

SEQ ID NO: 166 - protein, 25 aa, Artificial Sequence
  synthesized
EVSMIVNSHKPQKNLDSIQVTKDLT

SEQ ID NO: 167 - protein, 25 aa, Artificial Sequence
  synthesized
SLKFFQKKETKRGLDFTDSQENEEK

SEQ ID NO: 168 - protein, 25 aa, Artificial Sequence
  synthesized
ASCGTEGFRNAKEGTGIAAQTAGIA

SEQ ID NO: 169 - protein, 25 aa, Artificial Sequence
  synthesized
LWPSDSAAPRLGSSEIMHAPRSRDR

SEQ ID NO: 170 - protein, 25 aa, Artificial Sequence
  synthesized
ATEFFEDDATLTPSFCLEDGPKRYT

SEQ ID NO: 171 - protein, 25 aa, Artificial Sequence
  synthesized
NSECASKDVLVKNLRAIDVKLNSDN

SEQ ID NO: 172 - protein, 25 aa, Artificial Sequence
  synthesized
MEQERNARYRTPRILEPTAFQEPPP

SEQ ID NO: 173 - protein, 25 aa, Artificial Sequence
  synthesized
PPLWEKPPLGSSSCMLSPPLGRTTT

SEQ ID NO: 174 - protein, 25 aa, Artificial Sequence
  synthesized
PTTTRGLWAISERERSMKAILSFAK

SEQ ID NO: 175 - protein, 25 aa, Artificial Sequence
  synthesized
IKQLNISHLLVPPGLAKPAHLYDKG

SEQ ID NO: 176 - protein, 25 aa, Artificial Sequence
  synthesized
CVEERICHLQREIAWLVQQLDDVHQ

SEQ ID NO: 177 - protein, 25 aa, Artificial Sequence
  synthesized
FGSVTNIPAKDAPPVEKIDTMLVLE

SEQ ID NO: 178 - protein, 25 aa, Artificial Sequence
  synthesized
LGLAFNLNPALTIIGKYFYKRRPLA

SEQ ID NO: 179 - protein, 25 aa, Artificial Sequence
  synthesized
SQLPHCPSVSPSTQPWTHPGQSQLF

SEQ ID NO: 180 - protein, 25 aa, Artificial Sequence
  synthesized
DFTCDDGNDDNSYSPLSRCPTECTC

SEQ ID NO: 181 - protein, 25 aa, Artificial Sequence
  synthesized
KHDENLYGPIVKRISFFDFFKHKYQ

SEQ ID NO: 182 - protein, 25 aa, Artificial Sequence
  synthesized
HLKALKCLSLVPAVMGQLKFNTSEE

SEQ ID NO: 183 - protein, 25 aa, Artificial Sequence
  synthesized
GASIFQSKESSSELSCDISVDDKYI

SEQ ID NO: 184 - protein, 25 aa, Artificial Sequence
  synthesized
YMRRPNPVSPYPISSHTSDIYGSTS

SEQ ID NO: 185 - protein, 25 aa, Artificial Sequence
  synthesized
FNHHPYPHNHYMADLHPAAGHQMNG

SEQ ID NO: 186 - protein, 25 aa, Artificial Sequence
  synthesized
KWGGCSEDIEFGEMVSREFADAREN

SEQ ID NO: 187 - protein, 15 aa, Artificial Sequence
  synthesized
CAASPIIQGAQKLVF

SEQ ID NO: 188 - protein, 15 aa, Artificial Sequence
  synthesized
CASKISGTGLYEQYF

SEQ ID NO: 189 - protein, 12 aa, Artificial Sequence
  synthesized
CAVRDRAYKYIF

SEQ ID NO: 190 - protein, 15 aa, Artificial Sequence
  synthesized
CASSPRGTSENEQFF

SEQ ID NO: 191 - protein, 12 aa, Artificial Sequence
  synthesized
CALPGNTGKLIF

SEQ ID NO: 192 - protein, 18 aa, Artificial Sequence
  synthesized
CASSQVTSGRGLYNEQFF

SEQ ID NO: 193 - protein, 11 aa, Artificial Sequence
  synthesized
CAVFTGNQFYF

SEQ ID NO: 194 - protein, 13 aa, Artificial Sequence
  synthesized
CASSQIGGYGYTF

SEQ ID NO: 195 - protein, 13 aa, Artificial Sequence
  synthesized
CASGSGGSYIPTF

SEQ ID NO: 196 - protein, 14 aa, Artificial Sequence
  synthesized
CATSRTSGSYEQFF

SEQ ID NO: 197 - protein, 13 aa, Artificial Sequence
  synthesized
CVVSPNTGNQFYF

SEQ ID NO: 198 - protein, 12 aa, Artificial Sequence
  synthesized
CASSLFGEGYTF

SEQ ID NO: 199 - protein, 14 aa, Artificial Sequence
  synthesized
CAGVREGGSEKLVF

SEQ ID NO: 200 - protein, 15 aa, Artificial Sequence
  synthesized
CASSKVVNSYNEQFF

SEQ ID NO: 201 - protein, 13 aa, Artificial Sequence
  synthesized
CVVSPNTGNQFYF

SEQ ID NO: 202 - protein, 15 aa, Artificial Sequence
  synthesized
CASGEGSGVSYEQYF

SEQ ID NO: 203 - protein, 16 aa, Artificial Sequence
  synthesized
CAVSTNSGGSNYKLTF

SEQ ID NO: 204 - protein, 15 aa, Artificial Sequence
  synthesized
CASSLNRGFSDTQYF

SEQ ID NO: 205 - protein, 14 aa, Artificial Sequence
  synthesized
CAAREYGTASKLTF

SEQ ID NO: 206 - protein, 14 aa, Artificial Sequence
  synthesized
CASSPGTGNQPQHF

SEQ ID NO: 207 - protein, 14 aa, Artificial Sequence
  synthesized
CAATLWGGGNKLTF

SEQ ID NO: 208 - protein, 12 aa, Artificial Sequence
  synthesized
CSATNGGTEAFF

SEQ ID NO: 209 - protein, 16 aa, Artificial Sequence
  synthesized
CAVRDSFTSGTYKYIF

SEQ ID NO: 210 - protein, 14 aa, Artificial Sequence
  synthesized
CASSWTGAPYEQYF

SEQ ID NO: 211 - protein, 12 aa, Artificial Sequence
  synthesized
CAGQSYTDKLIF

SEQ ID NO: 212 - protein, 14 aa, Artificial Sequence
  synthesized
CSVEGTGEYNEQFF

SEQ ID NO: 213 - protein, 15 aa, Artificial Sequence
  synthesized
CAVRPSTGTASKLTF

SEQ ID NO: 214 - protein, 19 aa, Artificial Sequence
  synthesized
CASSPGTSGRPFPTDTQYF

SEQ ID NO: 215 - protein, 14 aa, Artificial Sequence
  synthesized
CAGLSGGSNYKLTF

SEQ ID NO: 216 - protein, 12 aa, Artificial Sequence
  synthesized
CAIRTESEKLFF

SEQ ID NO: 217 - protein, 15 aa, Artificial Sequence
  synthesized
CALSEATGSARQLTF

SEQ ID NO: 218 - protein, 14 aa, Artificial Sequence
  synthesized
CASSWFSANDEQFF

SEQ ID NO: 219 - protein, 13 aa, Artificial Sequence
  synthesized
CAASGTSGSRLTF

SEQ ID NO: 220 - protein, 12 aa, Artificial Sequence
  synthesized
CSANRGNYGYTF

SEQ ID NO: 221 - protein, 13 aa, Artificial Sequence
  synthesized
CAVNIQAGTALIF

SEQ ID NO: 222 - protein, 17 aa, Artificial Sequence
  synthesized
CASSDRSPGSGLETQYF

SEQ ID NO: 223 - protein, 14 aa, Artificial Sequence
  synthesized
CAVISSGSARQLTF

SEQ ID NO: 224 - protein, 12 aa, Artificial Sequence
  synthesized
CASSLVRNEQFF

SEQ ID NO: 225 - protein, 15 aa, Artificial Sequence
  synthesized
CAVRAPGGGADGLTF

SEQ ID NO: 226 - protein, 14 aa, Artificial Sequence
  synthesized
CASSFWREHNEQFF

SEQ ID NO: 227 - protein, 16 aa, Artificial Sequence
  synthesized
CAASANYGGSQGNLIF

SEQ ID NO: 228 - protein, 14 aa, Artificial Sequence
  synthesized
CSARAGRPPNAQYF

SEQ ID NO: 229 - protein, 12 aa, Artificial Sequence
  synthesized
CAMFGGYNKLIF

SEQ ID NO: 230 - protein, 15 aa, Artificial Sequence
  synthesized
CASSLNGQNTGELFF

SEQ ID NO: 231 - protein, 13 aa, Artificial Sequence
  synthesized
CALSSGSSYKLIF

SEQ ID NO: 232 - protein, 13 aa, Artificial Sequence
  synthesized
CASSLDGNSPLHF

SEQ ID NO: 233 - protein, 16 aa, Artificial Sequence
  synthesized
CAVSDSGSGGYQKVTF

SEQ ID NO: 234 - protein, 17 aa, Artificial Sequence
  synthesized
CATSDSGLAWNTGELFF

SEQ ID NO: 235 - protein, 11 aa, Artificial Sequence
  synthesized
CAEESGQNFVF

SEQ ID NO: 236 - protein, 15 aa, Artificial Sequence
  synthesized
CASSVGRVSTDTQYF

SEQ ID NO: 237 - protein, 13 aa, Artificial Sequence
  synthesized
CAVGSGNTGKLIF

SEQ ID NO: 238 - protein, 16 aa, Artificial Sequence
  synthesized
CASTRTSGRPNNEQFF

SEQ ID NO: 239 - protein, 11 aa, Artificial Sequence
  synthesized
CLVDNDYKLSF

SEQ ID NO: 240 - protein, 16 aa, Artificial Sequence
  synthesized
CAWSVQGDRAYYEQYF

SEQ ID NO: 241 - protein, 13 aa, Artificial Sequence
  synthesized
CAVCAGTASKLTF

SEQ ID NO: 242 - protein, 15 aa, Artificial Sequence
  synthesized
CASSPRPAGDNEQYF

SEQ ID NO: 243 - protein, 14 aa, Artificial Sequence
  synthesized
CAMTLTGGGNKLTF

SEQ ID NO: 244 - protein, 15 aa, Artificial Sequence
  synthesized
CASSQEDRSGNIQYF

SEQ ID NO: 245 - protein, 16 aa, Artificial Sequence
  synthesized
CATELVVGTSYGKLTF

SEQ ID NO: 246 - protein, 13 aa, Artificial Sequence
  synthesized
CASGLVPGEKLFF

SEQ ID NO: 247 - protein, 13 aa, Artificial Sequence
  synthesized
CATPDRGGFKTIF

SEQ ID NO: 248 - protein, 14 aa, Artificial Sequence
  synthesized
CASSLAGDMTGYTF

SEQ ID NO: 249 - protein, 17 aa, Artificial Sequence
  synthesized
CAGPSNAGGTSYGKLTF

SEQ ID NO: 250 - protein, 13 aa, Artificial Sequence
  synthesized
CASSPRTHGSYTF

SEQ ID NO: 251 - protein, 18 aa, Artificial Sequence
  synthesized
CAYRSWVRGIQGAQKLVF

SEQ ID NO: 252 - protein, 15 aa, Artificial Sequence
  synthesized
CASSPGQGARTEAFF

SEQ ID NO: 253 - protein, 10 aa, Artificial Sequence
  synthesized
CAYLVDKIIF

SEQ ID NO: 254 - protein, 14 aa, Artificial Sequence
  synthesized
CASSPTGGPYEQYF

SEQ ID NO: 255 - protein, 14 aa, Artificial Sequence
  synthesized
CAVILTGGGNKLTF

SEQ ID NO: 256 - protein, 13 aa, Artificial Sequence
  synthesized
CASSDSGTDTQYF

SEQ ID NO: 257 - protein, 15 aa, Artificial Sequence
  synthesized
CGTIVGGTSYGKLTF

SEQ ID NO: 258 - protein, 16 aa, Artificial Sequence
  synthesized
CASSFLDWEANYGYTF

SEQ ID NO: 259 - protein, 13 aa, Artificial Sequence
  synthesized
CAERDTNAGKSTF

SEQ ID NO: 260 - protein, 14 aa, Artificial Sequence
  synthesized
CASSLAPGDTEAFF

SEQ ID NO: 261 - protein, 14 aa, Artificial Sequence
  synthesized
CAEALSGGYNKLIF

SEQ ID NO: 262 - protein, 13 aa, Artificial Sequence
  synthesized
CASSFTGETEAFF

SEQ ID NO: 263 - protein, 12 aa, Artificial Sequence
  synthesized
CAPSNSGYALNF

SEQ ID NO: 264 - protein, 16 aa, Artificial Sequence
  synthesized
CASSLPGRRNTGELFF

SEQ ID NO: 265 - protein, 12 aa, Artificial Sequence
  synthesized
CVLDSGNTPLVF

SEQ ID NO: 266 - protein, 14 aa, Artificial Sequence
  synthesized
CASSPVGPSYEQYF

SEQ ID NO: 267 - protein, 14 aa, Artificial Sequence
  synthesized
CAVSDRANDYKLSF

SEQ ID NO: 268 - protein, 15 aa, Artificial Sequence
  synthesized
CASRPFRDSNQPQHF

SEQ ID NO: 269 - protein, 13 aa, Artificial Sequence
  synthesized
CAAAATNDYKLSF

SEQ ID NO: 270 - protein, 13 aa, Artificial Sequence
  synthesized
CASRTTGDYGYTF

SEQ ID NO: 271 - protein, 16 aa, Artificial Sequence
  synthesized
CLVGDPPLGGGNKLTF

SEQ ID NO: 272 - protein, 13 aa, Artificial Sequence
  synthesized
CASSFSRGGEAFF

SEQ ID NO: 273 - protein, 14 aa, Artificial Sequence
  synthesized
CAEGVYNQGGKLIF

SEQ ID NO: 274 - protein, 13 aa, Artificial Sequence
  synthesized
CASSLAVGGEQYF

SEQ ID NO: 275 - protein, 14 aa, Artificial Sequence
  synthesized
CAMRPHQAGTALIF

SEQ ID NO: 276 - protein, 15 aa, Artificial Sequence
  synthesized
CAVGGGPGTNYGYTF

SEQ ID NO: 277 - protein, 14 aa, Artificial Sequence
  synthesized
CALSGWGSGNQFYF

SEQ ID NO: 278 - protein, 16 aa, Artificial Sequence
  synthesized
CASSLTGTDNTGELFF

SEQ ID NO: 279 - protein, 14 aa, Artificial Sequence
  synthesized
CAMSAPYRDDKIIF

SEQ ID NO: 280 - protein, 15 aa, Artificial Sequence
  synthesized
CASSSDMTESYEQYF

SEQ ID NO: 281 - protein, 12 aa, Artificial Sequence
  synthesized
CAEKKGFQKLVF

SEQ ID NO: 282 - protein, 15 aa, Artificial Sequence
  synthesized
CASSPGEGGGTEAFF

SEQ ID NO: 283 - protein, 8 aa, Artificial Sequence
  synthesized
CAASKVTF

SEQ ID NO: 284 - protein, 14 aa, Artificial Sequence
  synthesized
CASTPGTGYSPLHF

SEQ ID NO: 285 - protein, 17 aa, Artificial Sequence
  synthesized
CAYRSPNYGGSQGNLIF

SEQ ID NO: 286 - protein, 15 aa, Artificial Sequence
  synthesized
CASSLGRGGIYEQYF

SEQ ID NO: 287 - protein, 11 aa, Artificial Sequence
  synthesized
CAAGDDYKLSF

SEQ ID NO: 288 - protein, 15 aa, Artificial Sequence
  synthesized
CSARDPRRTNTEAFF

SEQ ID NO: 289 - protein, 15 aa, Artificial Sequence
  synthesized
CAGASNAGNNRKLIW

SEQ ID NO: 290 - protein, 16 aa, Artificial Sequence
  synthesized
CASSLLLARRSDTQYF

SEQ ID NO: 291 - protein, 13 aa, Artificial Sequence
  synthesized
CVLPPAAGNKLTF

SEQ ID NO: 292 - protein, 14 aa, Artificial Sequence
  synthesized
CASSEQGSGNTIYF

SEQ ID NO: 293 - protein, 15 aa, Artificial Sequence
  synthesized
CAVRRDGGSQGNLIF

SEQ ID NO: 294 - protein, 14 aa, Artificial Sequence
  synthesized
CASTPRGKGEKLFF

SEQ ID NO: 295 - protein, 15 aa, Artificial Sequence
  synthesized
CALGRRGGATNKLIF

SEQ ID NO: 296 - protein, 15 aa, Artificial Sequence
  synthesized
CASSTGLAGQETQYF

SEQ ID NO: 297 - protein, 14 aa, Artificial Sequence
  synthesized
CAVESGGSNYKLTF

SEQ ID NO: 298 - protein, 14 aa, Artificial Sequence
  synthesized
CASSLDWENTEAFF

SEQ ID NO: 299 - protein, 11 aa, Artificial Sequence
  synthesized
CARYSGYALNF

SEQ ID NO: 300 - protein, 15 aa, Artificial Sequence
  synthesized
CASSPELADYNEQFF

SEQ ID NO: 301 - protein, 16 aa, Artificial Sequence
  synthesized
CAVKSHRGTSYGKLTF

SEQ ID NO: 302 - protein, 12 aa, Artificial Sequence
  synthesized
CASSFGPGELFF

SEQ ID NO: 303 - protein, 16 aa, Artificial Sequence
  synthesized
CAASPRIYNQGGKLIF

SEQ ID NO: 304 - protein, 15 aa, Artificial Sequence
  synthesized
CASSAVDRPTGELFF

SEQ ID NO: 305 - protein, 14 aa, Artificial Sequence
  synthesized
CAVRGMDSNYQLIW

SEQ ID NO: 306 - protein, 14 aa, Artificial Sequence
  synthesized
CASSLVGGSYEQYF

(19) United States
(12) Patent Application Publication
(10) Pub. No.: US 2024/0293454 A1
(43) Pub. Date: Sep. 5, 2024

(54) TCR-T CELL FOR KILLING TUMORS, AND PREPARATION METHOD THEREFOR AND USE THEREOF

(71) Applicant: GUANGZHOU FINELMMUNE BIOTECHNOLOGY CO., LTD., Guangzhou City, Guangdong (CN)

(72) Inventors: Haiping LIU, Guangzhou City, Guangdong (CN); Xiang LI, Guangzhou City, Guangdong (CN); You LI, Guangzhou City, Guangdong (CN); Chaochao BIAN, Guangzhou City, Guangdong (CN)

(21) Appl. No.: 18/016,293

(22) PCT Filed: Jun. 2, 2021
(86) PCT No.: PCT/CN2021/097813

§ 371 Date: Jan. 13, 2023

(30) Foreign Application Priority Data
Jul. 14, 2020 (CN) 202010674259.X

(51) Int. Cl. A61K 35/17; A61K 39/00; A61P 35/00; C07K 14/47; C07K 14/52; C07K 14/705; C12N 5/0783; C12N 15/86; C12Q 1/6841; C12Q 1/6869; C12Q 1/6886; G01N 33/50

(52) U.S. Cl. CPC A61K 35/17; A61K 39/4611; A61K 39/4632; A61K 39/464411; A61K 39/464417; A61K 39/464442; A61P 35/00; C07K 14/47; C07K 14/521; C07K 14/70521; C07K 14/70575; C07K 14/70596; C12N 5/0636; C12N 15/86; C12Q 1/6841; C12Q 1/6869; C12Q 1/6886; G01N 33/505; C12N 2740/15043; C12Q 2600/158; G01N 2333/7051

(57) ABSTRACT

The present disclosure provides a TCR-T cell for tumor-killing, wherein the TCR-T cell is a T cell carrying a TCR that recognizes a tumor antigen, and the TCR in the TCR-T cell is derived from any one or more of the following T cells: 1) a CD4 T cell expressing one or more of TNFRSF18, CXCL13, TNFRSF4, TNFSF8, ENTPD1, ACP5, LAYN, TNFRSF9, CTLA4, CD200 and TIGIT genes in the tumor; and 2) a CD8 T cell expressing one or more of TNFRSF18, CXCL13, CXCR6, GALNT2, ENTPD1, ACP5, HAVCR2, LAYN, TNFRSF9, CTLA4 and CD109 genes in the tumor. The TCR-T cell according to the present disclosure can be effectively applied to the treatment of a tumor, especially in an immunotherapy.

### CROSS-REFERENCE TO RELATED APPLICATIONS

The present application is a national phase entry under 35 USC § 371 of International Application PCT/CN2021/097813, filed Jun. 2, 2021, which claims the benefit of and priority to Chinese Patent Application No. 202010674259X, filed Jul. 14, 2020, the entire disclosures of which are incorporated herein by reference.

### INCORPORATION BY REFERENCE

This application includes a sequence listing in computer readable form (a “txt” file) that is submitted herewith on ASCII text file named 18016293 Sequence Listing, created on Jul. 12, 2023 and 105,152 bytes in size. This sequence listing is incorporated by reference herein.

### TECHNICAL FIELD

The present disclosure relates to the technical field of immunotherapy, in particular to a TCR-T cell for tumor-killing, a preparation method and use thereof.

### BACKGROUND

T cells recognize antigens presented by histocompatibility molecules (Human Leukocyte Antigen, HLA) on the surface of target cells via T Cell Receptors (TCR) on the surface thereof, thus achieving direct attack and killing of the target cells. Traditional TCR-T cell therapy for recognizing tumor antigens are directed at known tumor antigens and involve cloning TCRs that can recognize these known antigens and determining the HLA subtypes corresponding thereto. A patient who expresses both the tumor antigens and HLA subtypes is then selected to prepare TCR-T cells for re-infusion therapy. However, currently known tumor antigens are few, and these antigens are only expressed in a small number of patients. Moreover, it is required to express the corresponding HLA molecules simultaneously, which leads to very few patients who can meet the both requirements. Most patients cannot be treated by such TCR-T cells that recognize the known tumor antigens.

In tumor cells, amino acid sequence variants caused by gene mutations are the main source of tumor antigens. However, since mutations in tumors are random, and these tumor antigens produced by random mutations are usually unique to each patient. That is to say, there are very few tumor antigens shared among the patients. Therefore, in order to achieve effective treatment for most patients, it is necessary to establish corresponding TCR-T cells that recognize the tumor antigens specific to each patient.

During the occurrence and development of tumors, T cells that recognize tumor antigens in the body will be activated by the tumor antigens and enter tumor tissues to expand and kill tumor cells in the tumors. Therefore, if it is possible to identify and isolate T cells that recognize tumor antigens from the tumors in patients, obtain the TCR sequences carried thereby and enable expression thereof in the peripheral blood T cells of the patients, a TCR-T cell for tumor antigen recognizing derived from the patients themselves, i.e. an individualized TCR-T cell, will be established for individualized tumor therapy.

However, tumors are interconnected with the lymphatic and blood systems between normal tissues and blood, and a large number of T cells that do not recognize tumor antigens enter the tumors through the circulation of the blood and lymphatic systems. Therefore, in addition to the T cells that recognize the tumor antigens, the tumor tissue also contains a large number of T cells that do not recognize tumor antigens from the adjacent tissues and blood, that is, there is a mixture of various T cells. To obtain the TCRs that can recognize tumor antigens from such mixed T cells, it is necessary to first determine, by tumor antigen stimulation, which T cells can recognize the tumor antigens and then obtain the TCR sequences carried by these T cells for individualized TCR-T cell therapy. In addition, after whether these T cells recognize tumor antigens is ascertained, the characteristics of gene expression of these T cells are analyzed, the differences of the molecular characteristics between the T cells that recognize the tumor antigens and that do not recognize are compared and identified to find out the molecular markers specific to the T cells that can recognize the tumor antigens, and by means of these specific molecular markers, T cells recognizing the tumor antigens in the tumors can be isolated and obtained for subsequent treatment.

Identifying whether a T cell recognizes a tumor antigen is a systematic project with complicated process and high technical requirements. First of all, it is necessary to isolate T cells from a tumor tissue and culture the T cells in vitro. In addition, it is also necessary to identify the tumor antigen in the tumor tissue and then stimulate the isolated T cells in vitro with the identified tumor antigen. Only tumor antigen stimulation is the direct approach to prove if a T cell can recognize the tumor antigen. Since the whole experimental process of identification is extremely complicated, along with the high technical difficulty, systematic identification and analysis of tumor antigens in T cells in a tumor tissue currently remains impossible. As a result, it is currently also impossible to quickly obtain T cells and TCRs that recognize tumor antigens from a tumor tissue for treatment.

### SUMMARY

In view of the above technical problems to be solved, an object of the present disclosure is to provide a technical solution that provides a recognition capacity to accurately and quickly analyze tumor antigens of a T cell, in which by comparing the gene expression between T cells that recognize a tumor antigen and that do not recognize, the molecular markers specific to the T cells that recognize the tumor antigen in a tumor tissue are found out, and by means of these molecular markers, quick separation of the tumor antigen-recognizing T cells can be achieved, thus quickly obtaining TCRs that recognizes the tumor antigens, so as to establish an individualized therapeutic technique with the TCR-T cells recognizing the tumor antigens.

In order to achieve the above object, the present disclosure provides a TCR-T cell for tumor-killing, wherein the TCR-T cell is a T cell carrying a TCR that recognizes a tumor antigen, and the TCR in the TCR-T cell is derived from any one or more of the following T cells:
1) a CD4 T cell expressing one or more of TNFRSF18, CXCL13, TNFRSF4, TNFSF8, ENTPD1, ACP5, LAYN, TNFRSF9, CTLA4, CD200 and TIGIT genes in the tumor; and
2) a CD8 T cell expressing one or more of TNFRSF18, CXCL13, CXCR6, GALNT2, ENTPD1, ACP5, HAVCR2, LAYN, TNFRSF9, CTLA4 and CD109 genes in the tumor.

The present disclosure further provides use of the TCR-T cell in the preparation of a medicine for treating a tumor.

Preferably, the tumor comprises but not limited to any one or more selected from the group consisting of lung cancer, melanoma, intestinal cancer, liver cancer, stomach cancer, breast cancer, cervical cancer, ovarian cancer, kidney cancer, bladder cancer and esophageal cancer.

The present disclosure further provides a method for preparing the TCR-T cell, comprising: identifying a TCR that recognizes a tumor antigen; and introducing one or more nucleotide sequences of the TCR that recognizes a tumor antigen into a T cell for expression to construct the TCR-T cell.

Preferably, the step of identifying the TCR that recognizes the tumor antigen comprises: by means of flow sorting, magnetic bead separation or tumor tissue in-situ sequencing, obtaining, from a tumor tissue, a T cell, which expresses any one or more of the markers of a CD4 T cell for tumor antigen recognition, selected from the group consisting of TNFRSF18, CXCL13, TNFRSF4, TNFSF8, ENTPD1, ACP5, LAYN, TNFRSF9, CTLA4, CD200 and TIGIT, and a TCR sequence carried thereby; and a T cell, which expresses any one or more of the markers of a CD8 T cell for tumor antigen recognition, selected from the group consisting of TNFRSF18, CXCL13, CXCR6, GALNT2, ENTPD1, ACP5, HAVCR2, LAYN, TNFRSF9, CTLA4 and CD109, and a TCR sequence carried thereby.

The present disclosure further provides a method for identifying T cell and TCR for tumor antigen recognition, comprising: establishing a TCR-expressing TCR-T cell by cloning a high-frequency TCR in a tumor tissue, performing in vitro tumor antigen stimulation by using antigen-presenting cells expressing a tumor antigen tandem gene, and identifying a TCR carried by a TCR-T cell that can be stimulated as the TCR for tumor antigen recognition, and the T cell carrying such TCR as the T cell for tumor antigen recognition,
wherein the TCR in the TCR-T cell is derived from any one or more of the following T cells:
1) CD4 T cell expressing one or more of TNFRSF18, CXCL13, TNFRSF4, TNFSF8, ENTPD1, ACP5, LAYN, TNFRSF9, CTLA4, CD200 and TIGIT genes in the tumor; and
2) CD8 T cell expressing one or more of TNFRSF18, CXCL13, CXCR6, GALNT2, ENTPD1, ACP5, HAVCR2, LAYN, TNFRSF9, CTLA4 and CD109 genes in the tumor.

The TCR-T cell according to the present disclosure can be effectively applied to the treatment of a tumor, especially in an immunotherapy.

The present disclosure further provides a pharmaceutical composition, comprising the TCR-T cell.

The present disclosure further provides a method for treating a tumor, comprising administering a therapeutic amount of the TCR-T cell to a patient in need thereof.

### BRIEF DESCRIPTION OF DRAWINGS

FIG. 1 shows the stimulation responses of high-frequency TCRs in CD4 T cells and CD8 T cells to tumor antigens of tumors.

FIG. 2 shows that TCR-T cells constructed with TCRs from a subset of tumor antigen-recognizing T cells can effectively treat the tumor (*P<0.05; **P<0.01).

FIGS. 3A-B show the molecular marker genes of a subset of tumor antigen-recognizing T cells, wherein FIG. 3A shows the difference of the specific gene expression in CD4 T cells and FIG. 3B shows the difference of the specific gene expression in CD8 T cells.

FIGS. 4A-B show that T cells sorted by molecular markers of tumor antigen-recognizing T cells can effectively recognize tumor antigens (*P<0.05; **P<0.01), wherein FIG. 3A represents CD4 T cells and FIG. 3B represents CD8 T cells.

### DETAILED DESCRIPTION

In order to better explain the object, technical solution and advantages of the present disclosure, the present disclosure will be further explained hereinafter with reference to exemplary implementations. It should be noted that for the sake of brevity and clarity, some conventional technical operation steps, reagents and instruments may not be described in detail in the following exemplary implementations, and it should be understood that these conventional technical operation steps, reagents and instruments are obvious to those of ordinary skill in the art unless otherwise specified.

The present disclosure will be further described hereinafter with reference to exemplary implementations, and the advantages and features of the present disclosure may become apparent upon reading the description. However, the implementations are merely illustrative and are not intended to limit the scope of the present disclosure. Those skilled in the art should understand that the details and forms of the technical solution according to the present disclosure can be modified or replaced without departing from the spirit and scope of the present disclosure, and these modifications and replacements are all within the scope of protection of the present disclosure.

Firstly, DNA was extracted from a tumor tissue using DNeasy Blood & Tissue Kit (Art. No. 69506) from Qiagen, and RNA was extracted from the tumor tissue using miRNeasy-Mini Kit (Art. No. 217004) from Qiagen. The obtained DNA and RNA samples were then sent to a sequencing service company for exon and RNA sequencing. Moreover, DNA extracted from peripheral blood leukocytes was sent to the sequencing service company for exon sequencing, as a reference gene sequence for detecting gene mutation in the tumor. By comparing the exon sequences of the tumor tissue and the peripheral blood leukocytes, the mutation sites and the corresponding genes in the tumor tissue were found. In addition, according to the amount of expression of the RNA of each mutant gene in the tumor tissue, the expression abundances of the mutant genes in the tumor were ranked, and the mutants with high expression in the tumor tissue were selected as tumor antigens. Then, a 25 amino acid epitope peptide centered on the mutation site and a new polypeptide produced by frameshift mutation were connected in series as a tandem tumor antigen (that is, comprising the selected mutant polypeptides which are connected in series), a gene encoding the connected tumor mutant antigen in series (the tandem tumor antigen) was synthesized and expressed in immortalized B cells from a patient infected by EBV virus, whereby antigen-presenting cells covering most of the tumor mutant antigens were established and could be used for tumor antigen stimulation in vitro, thereby solving the difficult problem of failing to effectively obtain tumor antigens for antigen presentation in traditional methods.

Secondly, T cells in the tumor were sorted by flow cell sorting technology, and then, each of the T cells were subjected to RNA sequencing. Firstly, the tumor tissue was cut into pieces smaller than 1 mm³ with a scalpel, and the pieces were then added to a 10-fold volume of tissue digestion solution (150 ml of RIPA 1640 medium, 3 ml of fetal bovine serum, 27 mg of collagenase, and 7.5 mg of DNAse) and digested at 37° C. for 2 h. After complete digestion, the digested solution was filtered through a 70 μM cell strainer, washed and resuspended with PBS to obtain a single-cell suspension. A fluorescent flow antibody (color scheme: CD45-APC-Cy7, CD3-APC, and CD19-PE) was added to the sample with the final concentration of 1:200; and after staining at 4° C. for 30 min, the sample was washed twice with PBS, and individual T lymphocytes (CD45+CD3+) were sorted into a 96-well plate by a flow cell sorter (model: BD Bioscience Arial III). Then, single-cell RNA reverse transcription and cDNA amplification were carried out in the 96-well plate, and a library was constructed and sent to the sequencing service company for sequencing. The sequencing platform was Illumina HiSeq X Ten. The sequencing results were analyzed by CellRanger software to obtain the levels of the gene expression in each of the T cells and the TCR sequences carried thereby.

Then, the gene sequences of each TCR were synthesized and constructed into a lentiviral expression vector. Then, 293T cells were used to package the virus, a supernatant of the virus was collected, and the virus was concentrated by high-speed centrifugation. After the virus was resuspended, it was mixed with peripheral blood lymphocytes activated by CD3/CD28 antibody for 3 days. Polybrene with a final concentration of 8 μg/ml was added, and the mixture was then added to a 24-well plate and centrifuged at 30° C. at 2000 rpm for 50 min for T cell infection. Then, the supernatant was removed, and RPMI medium containing 10% FBS and 200 ng/ml IL2 was added for culture. A TCR-T cell line was established for each TCR by lentivirus centrifugal infection method, so as to use in vitro for long term and solve the difficult problem of tumor T cell culture in vitro in traditional identification methods.

After the establishment of the above two technical methods, the identification and analysis of T cells for tumor antigen recognition were carried out on a plurality of tumor tissues of different tumor types, including tumor types such as lung cancer, melanoma, intestinal cancer, liver cancer, stomach cancer, breast cancer, cervical cancer, ovarian cancer, kidney cancer, bladder cancer and esophageal cancer.

The representative results from three lung cancer tissues are shown below.

Fresh tumor (lung cancer) tissues (Tumor-1, Tumor-2, and Tumor-3) were obtained, and divided into three portions. Among them, the first portions were used to construct a PDX (Patient-Derived Xenograft) tumor model in an immunodeficient NSG mouse. Firstly, the tumor tissues were cut into pieces of about 1 mm³ with sterile scissor and then subcutaneously inoculated into the left groin of the immunodeficient NSG mouse with trocar. After tumor was formed, it was cut into small pieces again and further inoculated into the NSG mouse. After tumor formation for three times, the tumor was cut into small pieces and frozen in liquid nitrogen for subsequent tumor treatment experiment. The second portions of tumor tissues were used to extract DNA and RNA, and the extracted DNA and RNA were then sent to the sequencing service company for exon and RNA sequencing. Moreover, the DNA extracted from peripheral blood leukocytes was sent to the sequencing service company for exon sequencing, as a reference gene sequence for detecting gene mutation in the tumor. The third portions of tumor tissues were dissociated into single cells in suspension according to the above experimental scheme, and single CD4 T cell and CD8 T cell were then sorted into 96-well plates by flow sorting. Then, these T cells were respectively subjected to RNA sequencing to obtain the TCR sequences of each cell and the RNA expression levels of most genes. According to the results of exon and RNA sequencing, the mutant genes in each tumor tissue and the expression levels of these mutant genes were obtained. Then, the genes expressing mutant sequences were genetically connected in an antigen tandem manner to obtain a synthesized gene, which was transduced into EBV-immortalized B cells obtained from a patient, whereby a B cell line presenting tumor mutant antigen was established.

The selected mutant antigens and the synthesized tandem antigen sequences are as shown in Tables 1 to 3. In addition, by analyzing the TCR sequences carried by each of the CD4 T cells and CD8 T cells, 10 TCRs with the highest frequencies, i.e. the most amplified TCRs, were selected separately from CD4 T cells and CD8 T cells of each tumor, and cloned and constructed into a lentivirus expression vector. The information of the selected TCRs is as shown in Table 4. The vector was then introduced into the corresponding peripheral blood T cells of patients via lentivirus to prepare TCR-T cells, and the TCR-T cell line was established for each TCR.

TABLE 1
Tumor mutations of Tumor-1 and tandem antigen sequences

| Gene | Reference | Mutation | Mutant | Mutant | Tandem antigen | SEQ |
|---|---|---|---|---|---|---|
| name | sequence | type | base | amino acid | sequence | ID NOS |
| SGK1 | NM_001291995 | Missense | G16A | E6K | MTVKTKAAKGTLTYSR | 7 |
| | | mutation | | | MR | |
| CPNE1 | NM_001198863 | Missense | G505A | D169N | KSDPFLEFFRQGNGKW | 8 |
| | | mutation | | | HLVYRSEVI | |
| TMCC1 | NM_001017395 | Missense | A278T | D93V | MDLESQNACAEIVGVP | 9 |
| | | mutation | | | THPTALNRV | |
| KRT7 | NM_005556 | Missense | A553C | N185H | VEDFKNKYEDEIHHRT | 10 |
| | | mutation | | | AAENEFVVL | |
| CFH | NM_000186 | Missense | G860A | G287E | GDYSPLRIKHRTEDEIT | 11 |
| | | mutation | | | YQCRNGFY | |
| FGFR2 | NM_001144914 | Missense | A1929T | E643D | VPSQRPTFKQLVDDLDR | 12 |
| | | mutation | | | ILTLTTNE | |
| ITGB6 | NM_001282354 | Missense | G1640A | R547Q | CIECHLSAAGQAQEECV | 13 |
| | | mutation | | | DKCKLAGA | |
| HIPK2 | NM_001113239 | Missense | G1477C | E493Q | MTTDLEGSDMLVQKAD | 14 |
| | | mutation | | | RREFIDLLK | |
| PAM | NM_000919 | Missense | C790G | Q264E | NGQWTLIGRQSPELPQA | 15 |
| | | mutation | | | FYPVGHPV | |
| WWP2 | NM_199424 | Missense | C388G | H130D | GGIAREWFFLLSDEVLN | 16 |
| | | mutation | | | PMYCLFEY | |
| GRB7 | NM_001030002 | Missense | C946T | R316W | KPNKLRNGHKGLWIFC | 17 |
| | | mutation | | | SEDEQSRTC | |
| PLXNA3 | NM_017514 | Missense | G2689A | E897K | PAEYISAERIVCKMEES | 18 |
| | | mutation | | | LVPSPPPG | |
| RAVER2 | NM_018211 | Missense | G626T | G209V | AKARLELLGRQLVASA | 19 |
| | | mutation | | | LFAQWMDVN | |
| WDFY4 | NM_020945 | Missense | C8010G | F2670L | LQGGSFDVADRMLHSV | 20 |
| | | mutation | | | KSTWESASR | |
| ZMYM3 | NM_001171162 | Missense | G980A | R327H | RTPLQKGQTAYQHKGL | 21 |
| | | mutation | | | PQLFCSSSC | |
| KIAA1109 | NM_015312 | Missense | A3680C | H1227P | TQHLSLQVPLRSPSSSSS | 22 |
| | | mutation | | | SEENSSS | |
| NUP188 | NM_015354 | Missense | G3416A | G1139E | VVRRQLFLDVLDETKA | 23 |
| | | mutation | | | LLLVPASVN | |
| PWP2 | NM_005049 | Missense | A2644C | T882P | MQYALAVSKQRGPKRS | 24 |
| | | mutation | | | LDPLGSEEE | |
| RCHY1 | NM_001278537 | Missense | A569C | K190T | GRSTVQFHILGMTCKIC | 25 |
| | | mutation | | | ESYNTAQA | |
| INSR | NM_001079817 | Missense | G4030A | E1344K | DGGSSLGFKRSYKEHIP | 26 |
| | | mutation | | | YTHMNGGK | |
| PTPN14 | NM_005401 | Missense | C2621T | S874L | KRPLMLAALNGLLVAR | 27 |
| | | mutation | | | VSGREENRV | |
| SMYD2 | NM_020197 | Missense | T983G | M328R | ELLEICELSQEKRSSVFE | 28 |
| | | mutation | | | DSNVYML | |
| ZNF579 | NM_152600 | Missense | G1588A | A530T | PQSPPAPPVFLSTSCFDS | 29 |
| | | mutation | | | QDHSAFE | |
| AKNA | NM_030767 | Missense | G3436A | E1146K | KSTERLPGEPRGKEQIV | 30 |
| | | mutation | | | PPGRQRAR | |
| ZC3H14 | NM_001160103 | Missense | C9G | I3M | MEMGTEISRKIRSAI | 31 |
| | | mutation | | | | |
| RASGRF1 | NM_153815 | Missense | C1342G | Q448E | MISHIIREIRQFEQTAYKI | 32 |
| | | mutation | | | EHQAKV | |
| TACC2 | NM_001291878 | Missense | T363G | I121M | ASSGTYNLDFDNMELV | 33 |
| | | mutation | | | DTFQTLEPR | |
| PIAS3 | NM_006099 | Missense | A562T | I188L | REVLPGAKCDYTLQVQ | 34 |
| | | mutation | | | LRFCLCETS | |
| RETSAT | NM_017750 | Missense | C1598T | A533V | LTNQFYLAAPRGVCYG | 35 |
| | | mutation | | | ADHDLGRLH | |
| POLR3B | NM_001160708 | Missense | C2633T | S878L | CPDIIMNPHGFPLRMTV | 36 |
| | | mutation | | | GKLIELLA | |
| HGF | NM_000601 | Missense | C1708A | L570M | DEKCKQVLNVSQMVY | 37 |
| | | mutation | | | GPEGSDLVLM | |
| RETSAT | NM_017750 | Missense | G1606A | G536R | QFYLAAPRGACYRADH | 38 |
| | | mutation | | | DLGRLHPCV | |
| AIMP1 | NM_001142415 | Missense | G654A | M218I | MVILLCNLKPAKIRGVL | 39 |
| | | mutation | | | SQAMVMCA | |
| ROBO1 | NM_001145845 | Missense | C2908G | P970A | DSNLTTYSRPGQATPYA | 40 |
| | | mutation | | | TTQLIQSN | |
| CUL7 | NM_001168370 | Missense | C3208T | R1070C | WPVFREQLCRHTCLFY | 41 |
| | | mutation | | | MVRAQAWSQ | |
| PAN2 | NM_001127460 | Missense | G1801T | D601Y | EASALGLILADSYEASG | 42 |
| | | mutation | | | KGNLARLI | |
| VPS13B | NM_017890 | Missense | A5215T | S1739C | EVNITTNLDFFLCVAQV | 43 |
| | | mutation | | | QLLHQLIV | |
| TMOD2 | NM_001142885 | Missense | G55C | E19Q | KELEKYKNIDEDQLLG | 44 |
| | | mutation | | | KLSEEELKQ | |
| UNC5C | NM_003728 | Deletion | 2551 _ | 851_851del | GPSAFSIPLPIRQKLCSSL | 45 |
| | | frame | 2553del | | DAPQTR | |
| | | shift | | | | |
| C8orf33 | NM_023080 | Deletion | 641 _ | 214_223del | SQRVCRPRSIWRAKFRF | 46 |
| | | frame | 667del | | NFF | |
| | | shift | | | | |
| DNAH10 | NM_207437 | Missense | G3067A | E1023K | FAAKKPPCVAYDKKLQ | 47 |
| | | mutation | | | FYSKIAYEV | |
| TMEM80 | NM_001042463 | Missense | G242A | R81K | GGAGKMAAPRRGKGSS | 48 |
| | | mutation | | | TVLSSVPLQ | |
| SEC24C | NM_198597 | Missense | G1403A | G468D | VPPQYFQHLDHTDKRV | 49 |
| | | mutation | | | DAYDRPELS | |
| ARMC9 | NM_001291656 | Missense | A724C | I242L | RYNKIQADYHNLLGVT | 50 |
| | | mutation | | | AELVDSLEA | |
| ALMS1 | NM_015120 | Missense | G9423C | L3141F | PSLPDSNTITQDFKTIPS | 51 |
| | | mutation | | | QNSQIVT | |
| PDZD2 | NM_178140 | Missense | A1504C | K502Q | ESPKQGSNKIKLQSRLS | 52 |
| | | mutation | | | GGVHRLES | |
| IPO8 | NM_001190995 | Missense | T393G | H131Q | QQAFNYLNQGVVQSIT | 53 |
| | | mutation | | | WKQMKPHIQ | |
| TTN | NM_003319 | Missense | A76952C | H25651P | ELSSLRYSSPQAPVKVE | 54 |
| | | mutation | | | ETRKDFRY | |
| HDAC5 | NM_001015053 | Missense | A101G | E34G | PRTSLHSIPVTVGVKPV | 55 |
| | | mutation | | | LPRAMPSS | |
| RAC3 | NM_001316307 | Missense | G484C | D162H | GGLCEIPGVLSPHPAGP | 56 |
| | | mutation | | | EDSV | |
| ARMCX1 | NM_016608 | Missense | G1138A | E380K | KVPSELISLFNKKWDRE | 57 |
| | | mutation | | | ILLNILTL | |
| DNAH9 | NM_001372 | Missense | G8743C | E2915Q | GEIPDLYSDDEVQNIISN | 58 |
| | | mutation | | | VRNEVKS | |
| FRG1 | NM_004477 | Missense | A55G | T19A | VKSTKLVLKGTKAKSK | 59 |
| | | mutation | | | KKKSKDKKR | |
| UBAP1 | NM_001171201 | Missense | C193G | R65G | TEPGRPAGASTFGLLRR | 60 |
| | | mutation | | | RQQRHSGT | |
| ANO1 | NM_018043 | Missense | G340A | E114K | PGKGASLDAGSGKPPM | 61 |
| | | mutation | | | DYHEDDKRF | |
| ZNF48 | NM_152652 | Missense | G353A | G118D | ASSDRAAVCGECDKSF | 62 |
| | | mutation | | | RQMSDLVKH | |
| SRPK3 | NM_001170760 | Missense | A625C | I209L | HGLDYLHTKCKILHTDI | 63 |
| | | mutation | | | KPENILLC | |
| CDH11 | NM_001308392 | Missense | T1937G | M646R | PSLMEPPSPREDRRLLY | 64 |
| | | mutation | | | LGFQLMLF | |
| KSR2 | NM_173598 | Missense | G2666A | R889K | ILLFCWAFEQEEKPTFT | 65 |
| | | mutation | | | KLMDMLEK | |
| ADRA1B | NM_000679 | Insertion | 1122_ | R374delinsR | LGCQCRGRGRRRRRRR | 66 |
| | | frame | 1123ins | RR | RRRRLGGCAY | |
| | | shift | CGCCGC | | | |

The nucleotide sequences of Tumor-tandem antigen genes are as shown in SEQ ID NO. 1 and SEQ ID NO. 2.

TABLE 2
Tumor mutations of Tumor-2 and tandem antigen sequences

| Gene | Reference | Mutation | Mutant | Mutant | Tandem antigen | SEQ |
|---|---|---|---|---|---|---|
| name | sequence | type | base | amino acid | sequence | ID NOS |
| PCBP2 | NM_001098620 | Missense | A220G | M74V | AGPTNAIFKAFAVIIDKL | 67 |
| | | mutation | | | EEDISSS | |
| CTNNB1 | NM_001098209 | Missense | T109C | S37P | WQQQSYLDSGIHPGAT | 68 |
| | | mutation | | | TTAPSLSGK | |
| ACKR1 | NM_001122951 | Missense | A893G | H298R | LLLNLAEALAILRCVAT | 69 |
| | | mutation | | | PLLLALFC | |
| PRRC2C | NM_015172 | Missense | G6857C | S2286T | APPIATGVSSSATGPSTA | 70 |
| | | mutation | | | NYNSFSS | |
| PAPSS1 | NM_005443 | Missense | G964A | G322S | LTATHEDKERLDSCTAF | 71 |
| | | mutation | | | ALMYEGRR | |
| HIST1H2AE | NM_021052 | Missense | T197C | L66P | AVLEYLTAEILEPAGNA | 72 |
| | | mutation | | | ARDNKKTR | |
| ATP5J | NM_001003696 | Missense | T125C | L42P | VAFNKELDPIQKPFVDK | 73 |
| | | mutation | | | IREYKSKR | |
| KPNB1 | NM_001276453 | Missense | G433A | E145K | QGIEFWSNVCDEKMDL | 74 |
| | | mutation | | | AIEASEAAE | |
| FBXW2 | NM_012164 | Missense | G1274A | G425E | SSFLAGEASWLNELDG | 75 |
| | | mutation | | | HNDTGLVFA | |
| ZNF592 | NM_014630 | Missense | A1240T | I414F | SKSPVGSPLGSAFAEAP | 76 |
| | | mutation | | | SEMPGDEV | |
| NPRL2 | NM_006545 | Missense | G256T | G86C | KKYSRNALLFNLCFVC | 77 |
| | | mutation | | | DAQAKTCAL | |
| SMARCA4 | NM_001128845 | Missense | C2729T | T910M | NTHYVAPRRLLLMGTP | 78 |
| | | mutation | | | LONKLPELW | |
| EP300 | NM_001429 | Deletion | 433delC | Q145fs | SPNMGMGTSGPNRVLR | 79 |
| | | frame | | | SQQV | |
| | | shift | | | | |
| MGA | NM_001080541 | Deletion | 4816delC | H1606fs | HPNGQIVQLLPLISFEAL | 80 |
| | | frame | | | IPSPTYSLSCFGTQGL | |
| | | shift | | | | |
| BACE2 | NM_012105 | Missense | C899T | A300V | LNLDCREYNADKVIVD | 81 |
| | | mutation | | | SGTTLLRLP | |
| WSB2 | NM_001278557 | Missense | T174A | F58L | TWSVAFSPDGSWLAWS | 82 |
| | | mutation | | | QGHCIVKLI | |
| NCBP1 | NM_002486 | Missense | C1070T | A357V | IPLNYHIVEVIFVELFQL | 83 |
| | | mutation | | | PAPPHID | |
| RALGAPB | NM_001282917 | Deletion | 2763_ | S921fs | LLGAFPSPSGPASVVL | 84 |
| | | frame | 2766del | | | |
| | | shift | | | | |
| EPB41 | NM_203343 | Missense | C1460T | A487V | SGRTQAQTRQASVLIDR | 85 |
| | | mutation | | | PAPHFERT | |
| PNPLA7 | NM_152286 | Missense | C3917G | P1306R | SSLRHRHPSLAFRKLSE | 86 |
| | | mutation | | | GSSDQDG | |
| C3orf58 | NM_001134470 | Missense | G163T | A55S | YFNAPWEKRVDLSWQL | 87 |
| | | mutation | | | MEIAEQLTN | |
| TMEM130 | NM_001134451 | Missense | A118G | N40D | AEFLVGDLVVTQDTSLP | 88 |
| | | mutation | | | WPSSYLTK | |
| GPR137 | NM_001170880 | Missense | C728G | A243G | SRACYNLTALALGPQSR | 89 |
| | | mutation | | | LDTFDYDW | |
| CASS4 | NM_001164115 | Missense | G976A | A326T | TKNAVLTYPSPATLGHL | 90 |
| | | mutation | | | QAEAEKLE | |
| MAP2K3 | NM_002756 | Missense | T361C | F121L | ICMELMDTSLDKLYRK | 91 |
| | | mutation | | | VLDKNMTIP | |
| RASGRP3 | NM_015376 | Missense | T1376C | V459A | IAANFPFLDSFCALDKD | 92 |
| | | mutation | | | QDGLISKD | |
| DGKD | NM_003648 | Missense | G1901T | R634L | SESFGVPKGRSQLKVSK | 93 |
| | | mutation | | | SPCEKLIS | |
| KMT2C | NM_170606 | Missense | G5053T | A1685S | TRVKQIAKLWRKSSSQE | 94 |
| | | mutation | | | RAPYVQKA | |
| ASRGL1 | NM_001083926 | Missense | C827G | A276G | GLIVVSKTGDWVGKWT | 95 |
| | | mutation | | | STSMPWAAA | |
| CDR2 | NM_001802 | Missense | C242A | A81E | QVELLRQMNEQHEKVY | 96 |
| | | mutation | | | EQLDVTARE | |
| NOM1 | NM_138400 | Missense | C1859G | P620R | RWWIVGSAWSGARMID | 97 |
| | | mutation | | | NSHHTHLQK | |
| GATA2 | NM_001145662 | Missense | T862A | S288T | SSFTPKQRSKARTCSEG | 98 |
| | | mutation | | | RECVNCGA | |
| CRACR2B | NM_001286606 | Missense | A145G | I49V | LFLLCDKEAKGFVTKH | 99 |
| | | mutation | | | DLQGLQSDL | |
| COL6A3 | NM_057166 | Missense | G3850A | A1284T | GGRSPTVRVSVVTNTPS | 100 |
| | | mutation | | | GPVEAFDF | |
| TMCC1 | NM_001017395 | Missense | C1739T | A580V | QQQVVQLEGLENVTAR | 101 |
| | | mutation | | | NLLGKLINI | |
| RSBN1 | NM_018364 | Insertion | 185dupT | V62fs | AAQVGAVRVVRAVGG | 102 |
| | | frame | | | AGGAGORGEGETSCWG | |
| | | shift | | | LPAGS | |
| OLFML2B | NM_001297713 | Missense | C13T | R5W | MAKPWLLVLYFALIVV | 103 |
| | | mutation | | | P | |
| SLC38A7 | NM_001308384 | Missense | G38T | W13L | MAQVSINNDYSELDLST | 104 |
| | | mutation | | | DAGERARL | |
| B3GALT6 | NM_080605 | Missense | C560T | S187L | PARRRRLYWGFFLGRG | 105 |
| | | mutation | | | RVKPGGRWR | |
| MYRIP | NM_001284426 | Missense | G580A | E194K | LAKPKSGTFQALKVASS | 106 |
| | | mutation | | | VASAYDEM | |
| PGBD5 | NM_001258311 | Missense | A1148G | N383S | SDCTGLPLSMLTSPATP | 107 |
| | | mutation | | | PARGQYQI | |
| DIS3L2 | NM_152383 | Missense | C2183T | A728V | AAALGYRERLDMVPDT | 108 |
| | | mutation | | | LQKQADHCN | |
| WNK4 | NM_032387 | Missense | G3232A | E1078K | TREALAESDRAAKGLG | 109 |
| | | mutation | | | AGVEEEGDD | |
| WBP4 | NM_007187 | Missense | A745C | I249L | EGGVSTETEKPKLKFKE | 110 |
| | | mutation | | | KNKNSDGG | |
| CTU2 | NM_001012759 | Missense | C443T | A148V | ILQATGFPWHVVVLEE | 111 |
| | | mutation | | | VFSLPPSVL | |
| KMT5A | NM_020382 | Missense | G496C | A166P | KQALKKPIKGKQPPRK | 112 |
| | | mutation | | | KAQGKTQQN | |
| DUSP8 | NM_004420 | Missense | G1855C | V619L | AALGKQASFSGSLEVIE | 113 |
| | | mutation | | | VS | |
| PCDHA2 | NM_018905 | Missense | G194T | R65L | GLELEELVPRLFLVASK | 114 |
| | | mutation | | | RHGDLLEV | |
| CCDC142 | NM_032779 | Missense | C32T | P11L | MAQASRSGSLLPLVIVP | 115 |
| | | mutation | | | PLRAQP | |
| CNTNAP3 | NM_033655 | Missense | A3367G | T1123A | EEAVVMVEVNQSAKK | 116 |
| | | mutation | | | QVILSSGTEF | |
| TREML1 | NM_001271807 | Missense | T134C | V45A | SILVQCHYRLQDAKAQ | 117 |
| | | mutation | | | KVWCRFLPE | |
| TMEM191C | NM_001207052 | Missense | T494C | L165P | ELSSQLFYYGGEPQSQK | 118 |
| | | mutation | | | STEQQLAA | |
| USH2A | NM_206933 | Deletion | 14970_ | T4990fs | DTTLYIPRTADKTLFPG | 119 |
| | | frame | 14971del | | HLHD | |
| | | shift | | | | |
| BARHL2 | NM_020063 | Missense | G836T | W279L | AALNLTDTQVKTLYQN | 120 |
| | | mutation | | | RRTKWKRQT | |
| CFHR1 | NM_002113 | Missense | G523C | E175Q | MSKYPSGERVRYQCRS | 121 |
| | | mutation | | | PYEMFGDEE | |
| POTEF | NM_001099771 | Missense | T632C | I211T | LNVLDNKKRTALTKAV | 122 |
| | | mutation | | | QCQEDECAL | |
| PP2D1 | NM_001252657 | Missense | T896A | L299H | AKAFWRMDRLLGHGR | 123 |
| | | mutation | | | KEVSRVQWSG | |
| TOPAZ1 | NM_001145030 | Missense | G3361A | G1121R | RPLCKFAHVPEQRDEK | 124 |
| | | mutation | | | VCMDVFKKY | |
| FGF5 | NM_004464 | Missense | G112A | D38N | LAPKGQPGPAATNRNP | 125 |
| | | mutation | | | RGSSSRQSS | |
| STPG2 | NM_174952 | Missense | G1293C | E431D | FVKASKRFEESKDITPG | 126 |
| | | mutation | | | PATYEISQ | |

The nucleotide sequences of Tumor-2 tandem antigen genes are as shown in SEQ ID NO. 3 and SEQ ID NO. 4.

TABLE 3
Tumor mutations of Tumor-3 and tandem antigen sequences

| Gene | Reference | Mutation | Mutant | Mutant amino | Tandem antigen | SEQ |
|---|---|---|---|---|---|---|
| name | sequence | type | base | acid | sequence | ID NOS |
| PSMA1 | NM_002786 | Missense | A767G | K256R | AQPAQPADEPAER | 127 |
| | | mutation | | | ADEPMEH | |
| VCP | NM_007126 | Missense | A1309G | I437V | KKMDLIDLEDETV | 128 |
| | | mutation | | | DAEVMNSLAVTM | |
| WNK1 | NM_014823 | Missense | G5025C | E1675D | GITIPGISSDVPDSA | 129 |
| | | mutation | | | HKTTASEAKS | |
| PABPC1 | NM_002568 | Missense | T1361C | I454T | ARPHPFQNMPGAT | 130 |
| | | mutation | | | RPAAPRPPFSTM | |
| HSPG2 | NM_001291860 | Missense | T839A | V280D | VTHAPQPLLPGSDR | 131 |
| | | mutation | | | PLPCGPQEAAC | |
| ATN1 | NM_001007026 | Missense | G2146A | A716T | GLPSLPPPPAAPTSG | 132 |
| | | mutation | | | PPLSATQIKQ | |
| TP53 | NM_001126115 | Missense | G128A | R43H | YKQSQHMTEVVRH | 133 |
| | | mutation | | | CPHHERCSDSDG | |
| PDE4DIP | NM_001002811 | Deletion | 1947delT | V649fs | LEKLRQRIHDKAVL | 134 |
| | | frame | | | WSGL | |
| | | shift | | | | |
| EPRS | NM_004446 | Missense | C4150G | R1384G | RDMKSCQFVAVRG | 135 |
| | | mutation | | | DTGEKLTVAENE | |
| PARP14 | NM_017554 | Missense | G3541A | A1181T | FLLHPSDHENIQTFS | 136 |
| | | mutation | | | DEFARRANGN | |
| METAP1 | NM_015143 | Missense | G961A | G321S | YAKNKAVGVMKS | 137 |
| | | mutation | | | SHVFTIEPMICEG | |
| IGSF3 | NM_001542 | Missense | G921T | Q307H | GEPVEFRCILEAHN | 138 |
| | | mutation | | | VPDRYFAVSWA | |
| LTBP1 | NM_001166264 | Missense | C3814A | Q1272K | LNGCENGRCVRVK | 139 |
| | | mutation | | | EGYTCDCFDGYH | |
| TPR | NM_003292 | Missense | A3830G | K1277R | KTETMNVVMETNR | 140 |
| | | mutation | | | MLREEKERLEQD | |
| KCTD3 | NM_016121 | Missense | G763C | E255Q | HGDKDKMVAVAS | 141 |
| | | mutation | | | QSSIILWSVQDGG | |
| LMBR1L | NM_001300751 | Missense | T565G | F189V | DKNKANRESLYDV | 142 |
| | | mutation | | | WEYYLPYLYSCI | |
| TSPAN15 | NM_012339 | Missense | C755T | T252M | GILLPQFLGVLLML | 143 |
| | | mutation | | | LYITRVEDIIM | |
| SEMA4A | NM_001193302 | Missense | G233T | R78L | MNNFLGSEPILMLT | 144 |
| | | mutation | | | LGSQPVLKTDN | |
| DNMIL | NM_001278466 | Missense | C616G | P206A | RNATGPRPALFVAE | 145 |
| | | mutation | | | VSFELLVKRQI | |
| RUBCN | NM_001145642 | Missense | A146G | D49G | LHVEKFISVHENGQ | 146 |
| | | mutation | | | SSADGASERAV | |
| FLT4 | NM_002020 | Missense | G3550T | V1184F | LLQGRGLQEEEEFC | 147 |
| | | mutation | | | MAPRSSQSSEE | |
| PPP4R3A | NM_001284281 | Missense | G421T | A141S | VILGMDDTQVRSS | 148 |
| | | mutation | | | ATDIFSYLVEYN | |
| MCRS1 | NM_001278341 | Missense | G331C | E111Q | DSKLKDMRDEVLQ | 149 |
| | | mutation | | | HELMVADRRQKR | |
| TMEM94 | NM_014738 | Missense | G2380A | A794T | CELPSTIPIKQNTRR | 150 |
| | | mutation | | | SSWSSDEGIG | |
| WDFY3 | NM_014991 | Missense | G5543A | R1848H | CTEAVFLLLGMLH | 151 |
| | | mutation | | | SMLTSPWQSEEE | |
| PROSER3 | NM_001039887 | Deletion | 653delC | S218fs | LKRSKASISSSSSSA | 152 |
| | | frame shift | | | PAMPALPHSPPAL | |
| | | | | | MASLPSRRPSSLTP | |
| | | | | | ARALAPGHPHPRH | |
| | | | | | QPRPRPPPLLQPQPP | |
| | | | | | PKHPFGQRMTFCTS | |
| | | | | | GGSGGSLNRLREA | |
| | | | | | RVTELGCRL | |
| MBTPS2 | NM_015884 | Missense | A468T | Q156H | QVVVPGINLPVNHL | 153 |
| | | mutation | | | TYFFTAVLISG | |
| ZBTB48 | NM_001278647 | Missense | T1397C | V466A | HIKAKHRNERPHA | 154 |
| | | mutation | | | CEFCSHAFTQKA | |
| STK16 | NM_001008910 | Missense | G139A | A47T | DLVEGLHDGHFYT | 155 |
| | | mutation | | | LKRILCHEQQDR | |
| TLR1 | NM_003263 | Missense | A7G | S3G | MTGIFHFAIIFMLIL | 156 |
| | | mutation | | | | |
| NOTCH2 | NM_024408 | Missense | C6765A | N2255K | RLHPVPVPADWMK | 157 |
| | | mutation | | | RMEVNETQYNEM | |
| NLRC5 | NM_032206 | Deletion | 1519_ | Q507fs | TSFCVCTGPGHQQ | 158 |
| | | frame | 1522del | | AMLSPTSACRSFLL | |
| | | shift | | | PCT | |
| ACAP1 | NM_014716 | Missense | G1624A | V542M | RGGRGRPRGQPPM | 159 |
| | | mutation | | | PPKPSIRPRPGS | |
| PIK3AP1 | NM_152309 | Missense | G2347T | A783S | MSLERPPRVPPRSA | 160 |
| | | mutation | | | SQRPPTRETFH | |
| MICAL3 | NM_015241 | Missense | A4597G | K1533E | PFADDVEDTYDDE | 161 |
| | | mutation | | | TEDSSLQEKFFT | |
| FYB | NM_001243093 | Insertion | 1002dupG | P335fs | FPKAPSKLTVGGA | 162 |
| | | frame | | | MGPKSGKGKGRQE | |
| | | shift | | | FSHPETEAIASLVY | |
| | | | | | LGSTSTKTQQTTKC | |
| PTBP1 | NM_002819 | Missense | C686T | A229V | TKNNQFQALLQYV | 163 |
| | | mutation | | | DPVSAQHAKLSL | |
| FILIP1L | NM_001282794 | Missense | C2551G | R851G | RPASPSAPLQDNGT | 164 |
| | | mutation | | | QGLINGALNKT | |
| SLC35A5 | NM_017945 | Missense | G1199A | R400H | ERIRDLSGNLWEHS | 165 |
| | | mutation | | | SGDGEELERLT | |
| CRYBG3 | NM_153605 | Missense | T3243G | N1081K | EVSMIVNSHKPQK | 166 |
| | | mutation | | | NLDSIQVTKDLT | |
| USP1 | NM_001017415 | Missense | C107G | A36G | SLKFFQKKETKRGL | 167 |
| | | mutation | | | DFTDSQENEEK | |
| MRPS11 | NM_176805 | Missense | A265G | K89E | ASCGTEGFRNAKE | 168 |
| | | mutation | | | GTGIAAQTAGIA | |
| LDLRAD4 | NM_001276251 | Missense | G88T | A30S | LWPSDSAAPRLGSS | 169 |
| | | mutation | | | EIMHAPRSRDR | |
| LDB2 | NM_001130834 | Missense | T200C | L67P | ATEFFEDDATLTPS | 170 |
| | | mutation | | | FCLEDGPKRYT | |
| CEP192 | NM_032142 | Missense | C1082A | T361N | NSECASKDVLVKN | 171 |
| | | mutation | | | LRAIDVKLNSDN | |
| PTK2B | NM_173175 | Missense | A2108G | K703R | MEQERNARYRTPRI | 172 |
| | | mutation | | | LEPTAFQEPPP | |
| CCDC191 | NM_020817 | Missense | G1462A | G488S | PPLWEKPPLGSSSC | 173 |
| | | mutation | | | MLSPPLGRTTT | |
| SHPRH | NM_001042683 | Missense | C3890G | T1297R | PTTTRGLWAISERE | 174 |
| | | mutation | | | RSMKAILSFAK | |
| TMEM2 | NM_001135820 | Missense | T3731C | L1244P | IKQLNISHLLVPPGL | 175 |
| | | mutation | | | AKPAHLYDKG | |
| ANKRD20A4 | NM_001098805 | Missense | A2222T | N741I | CVEERICHLQREIA | 176 |
| | | mutation | | | WLVQQLDDVHQ | |
| ANAPCI1 | NM_022662 | Missense | G1438C | A480P | FGSVTNIPAKDAPP | 177 |
| | | mutation | | | VEKIDTMLVLE | |
| SLC16A1 | NM_001166496 | Missense | G402A | M134I | LGLAFNLNPALTIIG | 178 |
| | | mutation | | | KYFYKRRPLA | |
| AOC3 | NM_001277731 | Missense | G142A | A48T | SQLPHCPSVSPSTQ | 179 |
| | | mutation | | | PWTHPGQSQLF | |
| SLIT2 | NM_001289135 | Missense | G2150A | C717Y | DFTCDDGNDDNSY | 180 |
| | | mutation | | | SPLSRCPTECTC | |
| KDELC1 | NM_024089 | Missense | A1058G | H353R | KHDENLYGPIVKRI | 181 |
| | | mutation | | | SFFDFFKHKYQ | |
| PRKRIR | NM_004705 | Missense | T1828G | S610A | HLKALKCLSLVPA | 182 |
| | | mutation | | | VMGQLKFNTSEE | |
| TLE1 | NM_001303103 | Missense | T2255A | V752E | GASIFQSKESSSELS | 183 |
| | | mutation | | | CDISVDDKYI | |
| TET2 | NM_001127208 | Missense | A4742T | N1581I | YMRRPNPVSPYPIS | 184 |
| | | mutation | | | SHTSDIYGSTS | |
| CITED2 | NM_001168388 | Missense | C406G | P136A | FNHHPYPHNHYMA | 185 |
| | | mutation | | | DLHPAAGHQMNG | |
| WNT3A | NM_033131 | Missense | G488A | G163E | KWGGCSEDIEFGE | 186 |
| | | mutation | | | MVSREFADAREN | |

The nucleotide sequences of Tumor-3 tandem antigen genes are as shown in SEQ ID NO. 5 and SEQ ID NO. 6.

TABLE 4
Information of amino acid sequences of TCR a and b chains

| | | | | b-V | b-D | b-J | |
|---|---|---|---|---|---|---|---|
| Tumor-1 | a-V gene | a-J gene | a-CDR3 | gene | gene | gene | b-CDR3 |
| CD4 TCR-1 | TRAV29DV5 | TRAJ54 | CAASPIIQGAQKL | TRBV19 | TRBD1 | TRBJ2-7 | CASKISGTGLYEQY |
| | | | VF (SEQ ID NO. | | | | F (SEQ ID NO. 188) |
| | | | 187) | | | | |
| CD4 TCR-2 | TRAV3 | TRAJ40 | CAVRDRAYKYIF | TRBV9 | TRBD2 | TRBJ2-1 | CASSPRGTSENEQF |
| | | | (SEQ ID NO. 189) | | | | F (SEQ ID NO. 190) |
| CD4 TCR-3 | TRAV21 | TRAJ37 | CALPGNTGKLIF | TRBV4-1 | TRBD2 | TRBJ2-1 | CASSQVTSGRGLY |
| | | | (SEQ ID NO. 191) | | | | NEQFF (SEQ ID NO. |
| | | | | | | | 192) |
| CD4 TCR-4 | TRAV22 | TRAJ49 | CAVFTGNQFYF | TRBV4-3 | TRBD1 | TRBJ1-2 | CASSQIGGYGYTF |
| | | | (SEQ ID NO. 193) | | | | (SEQ ID NO. 194) |
| CD4 TCR-5 | TRAV12-1 | TRAJ6 | CASGSGGSYIPTF | TRBV15 | TRBD2 | TRBJ2-1 | CATSRTSGSYEQFF |
| | | | (SEQ ID NO. 195) | | | | (SEQ ID NO. 196) |
| CD4 TCR-6 | TRAV8-2 | TRAJ49 | CVVSPNTGNQFYF | TRBV7-3 | None | TRBJ1-2 | CASSLFGEGYTF |
| | | | (SEQ ID NO. 197) | | | | (SEQ ID NO. 198) |
| CD4 TCR-7 | TRAV27 | TRAJ57 | CAGVREGGSEKLV | TRBV5-1 | None | TRBJ2-1 | CASSKVVNSYNEQ |
| | | | F (SEQ ID NO. 199) | | | | FF (SEQ ID NO. 200) |
| CD4 TCR-8 | TRAV8-2 | TRAJ49 | CVVSPNTGNQFYF | TRBV11- | TRBD2 | TRBJ2-7 | CASGEGSGVSYEQ |
| | | | (SEQ ID NO. 201) | 2 | | | YF (SEQ ID NO. |
| | | | | | | | 202) |
| CD4 TCR-9 | TRAV8-6 | TRAJ53 | CAVSTNSGGSNYK | TRBV6-5 | TRBD1 | TRBJ2-3 | CASSLNRGFSDTQ |
| | | | LTF (SEQ ID NO. | | | | YF (SEQ ID NO. |
| | | | 203) | | | | 204) |
| CD4 TCR-10 | TRAV13-1 | TRAJ44 | CAAREYGTASKLT | TRBV5-8 | TRBD2 | TRBJ1-5 | CASSPGTGNQPQH |
| | | | F (SEQ ID NO. 205) | | | | F (SEQ ID NO. 206) |
| | | | | b-V | b-D | b-J | |
| Tumor-1 | a-V gene | a-J gene | a-CDR3 | gene | gene | gene | b-CDR3 |
| CD8 TCR-1 | TRAV13-1 | TRAJ10 | CAATLWGGGNKL | TRBV20- | TRBD1 | TRBJ1-1 | CSATNGGTEAFF |
| | | | TF (SEQ ID NO. | 1 | | | (SEQ ID NO. 208) |
| | | | 207) | | | | |
| CD8 TCR-2 | TRAV3 | TRAJ40 | CAVRDSFTSGTYK | TRBV27 | TRBD1 | TRBJ2-7 | CASSWTGAPYEQY |
| | | | YIF (SEQ ID NO. | | | | F (SEQ ID NO. 210) |
| | | | 209) | | | | |
| CD8 TCR-3 | TRAV35 | TRAJ34 | CAGQSYTDKLIF | TRBV29- | TRBD1 | TRBJ2-1 | CSVEGTGEYNEQF |
| | | | (SEQ ID NO. 211) | 1 | | | F (SEQ ID NO. 212) |
| CD8 TCR-4 | TRAV3 | TRAJ44 | CAVRPSTGTASKL | TRBV5-1 | TRBD2 | TRBJ2-3 | CASSPGTSGRPFPT |
| | | | TF (SEQ ID NO. | | | | DTQYF (SEQ ID NO. |
| | | | 213) | | | | 214) |
| CD8 TCR-5 | TRAV25 | TRAJ53 | CAGLSGGSNYKLT | TRBV10- | TRBD1 | TRBJ1-4 | CAIRTESEKLFF |
| | | | F (SEQ ID NO. 215) | 3 | | | (SEQ ID NO. 216) |
| CD8 TCR-6 | TRAV19 | TRAJ22 | CALSEATGSARQL | TRBV27 | TRBD2 | TRBJ2-1 | CASSWFSANDEQF |
| | | | TF (SEQ ID NO. | | | | F (SEQ ID NO. 218) |
| | | | 217) | | | | |
| CD8 TCR-7 | TRAV29DV5 | TRAJ58 | CAASGTSGSRLTF | TRBV20-1 | TRBD1 | TRBJ1-2 | CSANRGNYGYTF |
| | | | (SEQ ID NO. 219) | | | | (SEQ ID NO. 220) |
| CD8 TCR-8 | TRAV12-2 | TRAJ15 | CAVNIQAGTALIF | TRBV19 | TRBD2 | TRBJ2-5 | CASSDRSPGSGLET |
| | | | (SEQ ID NO. 221) | | | | QYF (SEQ ID NO. |
| | | | | | | | 222) |
| CD8 TCR-9 | TRAV12-2 | TRAJ22 | CAVISSGSARQLTF | TRBV7-2 | None | TRBJ2-1 | CASSL VRNEQFF |
| | | | (SEQ ID NO. 223) | | | | (SEQ ID NO. 224) |
| CD8 TCR-10 | TRAV3 | TRAJ45 | CAVRAPGGGADG | TRBV5-8 | TRBD2 | TRBJ2-1 | CASSFWREHNEQF |
| | | | LTF (SEQ ID NO. | | | | F (SEQ ID NO. 226) |
| | | | 225) | | | | |
| | | | | b-V | b-D | b-J | |
| Tumor-2 | a-V gene | a-J gene | a-CDR3 | gene | gene | gene | b-CDR3 |
| CD4 TCR-1 | TRAV29DV5 | TRAJ42 | CAASANYGGSQG | TRBV20- | TRBD2 | TRBJ2-7 | CSARAGRPPNAQY |
| | | | NLIF (SEQ ID NO. | 1 | | | F (SEQ ID NO. 228) |
| | | | 227) | | | | |
| CD4 TCR-2 | TRAV12-3 | TRAJ4 | CAMFGGYNKLIF | TRBV7-3 | None | TRBJ2-2 | CASSLNGQNTGEL |
| | | | (SEQ ID NO. 229) | | | | FF (SEQ ID NO. 230) |
| CD4 TCR-3 | TRAV9-2 | TRAJ12 | CALSSGSSYKLIF | TRBV12- | None | TRBJ1-6 | CASSLDGNSPLHF |
| | | | (SEQ ID NO. 231) | 3 | | | (SEQ ID NO. 232) |
| CD4 TCR-4 | TRAV8-4 | TRAJ13 | CAVSDSGSGGYQ | TRBV24- | TRBD2 | TRBJ2-2 | CATSDSGLAWNTG |
| | | | KVTF (SEQ ID NO. | 1 | | | ELFF (SEQ ID NO. |
| | | | 233) | | | | 234) |
| CD4 TCR-5 | TRAV5 | TRAJ26 | CAEESGQNFVF | TRBV9 | TRBD2 | TRBJ2-3 | CASSVGRVSTDTQ |
| | | | (SEQ ID NO. 235) | | | | YF (SEQ ID NO. |
| | | | | | | | 236) |
| CD4 TCR-6 | TRAV21 | TRAJ37 | CAVGSGNTGKLIF | TRBV6-5 | TRBD2 | TRBJ2-1 | CASTRTSGRPNNE |
| | | | (SEQ ID NO. 237) | | | | QFF (SEQ ID NO. |
| | | | | | | | 238) |
| CD4 TCR-7 | TRAV4 | TRAJ20 | CLVDNDYKLSF | TRBV30 | TRBD1 | TRBJ2-7 | CAWSVQGDRAYY |
| | | | (SEQ ID NO. 239) | | | | EQYF (SEQ ID NO. |
| | | | | | | | 240) |
| CD4 TCR-8 | TRAV21 | TRAJ44 | CAVCAGTASKLTF | TRBV12- | TRBD2 | TRBJ2-7 | CASSPRPAGDNEQ |
| | | | (SEQ ID NO. 241) | 3 | | | YF (SEQ ID NO. |
| | | | | | | | 242) |
| CD4 TCR-9 | TRAV12-3 | TRAJ10 | CAMTLTGGGNKL | TRBV4-2 | TRBD1 | TRBJ2-4 | CASSQEDRSGNIQY |
| | | | TF (SEQ ID NO. | | | | F (SEQ ID NO. 244) |
| | | | 243) | | | | |
| CD4 TCR-10 | TRAV17 | TRAJ52 | CATELVVGTSYGK | TRBV12- | TRBD2 | TRBJ1-4 | CASGLVPGEKLFF |
| | | | LTF (SEQ ID NO. | 5 | | | (SEQ ID NO. 246) |
| | | | 245) | | | | |
| | | | | b-V | b-D | b-J | |
| Tumor-2 | a-V gene | a-J gene | a-CDR3 | gene | gene | gene | b-CDR3 |
| CD8 TCR-1 | TRAV17 | TRAJ9 | CATPDRGGFKTIF | TRBV5-1 | TRBD1 | TRBJ1-2 | CASSLAGDMTGYT |
| | | | (SEQ ID NO. 247) | | | | F (SEQ ID NO. 248) |
| CD8 TCR-2 | TRAV25 | TRAJ52 | CAGPSNAGGTSYG | TRBV6-5 | TRBD1 | TRBJ1-2 | CASSPRTHGSYTF |
| | | | KLTF (SEQ ID NO. | | | | (SEQ ID NO. 250) |
| | | | 249) | | | | |
| CD8 TCR-3 | TRAV38- | TRAJ54 | CAYRSWVRGIQG | TRBV5-1 | TRBD1 | TRBJ1-1 | CASSPGQGARTEA |
| | 2DV8 | | AQKLVF (SEQ ID | | | | FF (SEQ ID NO. 252) |
| | | | NO. 251) | | | | |
| CD8 TCR-4 | TRAV38- | TRAJ30 | CAYLVDKIIF (SEQ | TRBV5-1 | TRBD1 | TRBJ2-7 | CASSPTGGPYEQYF |
| | 2DV8 | | ID NO. 253) | | | | (SEQ ID NO. 254) |
| CD8 TCR-5 | TRAV20 | TRAJ10 | CAVILTGGGNKLT | TRBV18 | TRBD1 | TRBJ2-3 | CASSDSGTDTQYF |
| | | | F (SEQ ID NO. 255) | | | | (SEQ ID NO. 256) |
| CD8 TCR-6 | TRAV12-1 | TRAJ52 | CGTIVGGTSYGKL | TRBV6-3 | TRBD2 | TRBJ1-2 | CASSFLDWEANYG |
| | | | TF (SEQ ID NO. | | | | YTF (SEQ ID NO. |
| | | | 257) | | | | 258) |
| CD8 TCR-7 | TRAV5 | TRAJ27 | CAERDTNAGKSTF | TRBV7-8 | None | TRBJ1-1 | CASSLAPGDTEAFF |
| | | | (SEQ ID NO. 259) | | | | (SEQ ID NO. 260) |
| CD8 TCR-8 | TRAV5 | TRAJ4 | CAEALSGGYNKLI | TRBV7-9 | TRBD1 | TRBJ1-1 | CASSFTGETEAFF |
| | | | F (SEQ ID NO. 261) | | | | (SEQ ID NO. 262) |
| CD8 TCR-9 | TRAV38- | TRAJ41 | CAPSNSGYALNF | TRBV12-4 | TRBD2 | TRBJ2-2 | CASSLPGRRNTGEL |
| | 2DV8 | | (SEQ ID NO. 263) | | | | FF (SEQ ID NO. 264) |
| CD8 TCR-10 | TRAV12-1 | TRAJ29 | CVLDSGNTPLVF | TRBV14 | None | TRBJ2-7 | CASSPVGPSYEQYF |
| | | | (SEQ ID NO. 265) | | | | (SEQ ID NO. 266) |
| | | | | b-V | b-D | b-J | |
| Tumor-3 | a-V gene | a-J gene | a-CDR3 | gene | gene | gene | b-CDR3 |
| CD4 TCR-1 | TRAV8-4 | TRAJ20 | CAVSDRANDYKL | TRBV6-5 | TRBD1 | TRBJ1-5 | CASRPFRDSNQPQ |
| | | | SF (SEQ ID NO. | | | | HF (SEQ ID NO. |
| | | | 267) | | | | 268) |
| CD4 TCR-2 | TRAV5 | TRAJ20 | CAAAATNDYKLSF | TRBV5-4 | TRBD1 | TRBJ1-2 | CASRTTGDYGYTF |
| | | | (SEQ ID NO. 269) | | | | (SEQ ID NO. 270) |
| CD4 TCR-3 | TRAV4 | TRAJ10 | CLVGDPPLGGGNK | TRBV7-3 | TRBD1 | TRBJ1-1 | CASSFSRGGEAFF |
| | | | LTF (SEQ ID NO. | | | | (SEQ ID NO. 272) |
| | | | 271) | | | | |
| CD4 TCR-4 | TRAV5 | TRAJ23 | CAEGVYNQGGKLI | TRBV7-8 | TRBD1 | TRBJ2-7 | CASSLAVGGEQYF |
| | | | F (SEQ ID NO. 273) | | | | (SEQ ID NO. 274) |
| CD4 TCR-5 | TRAV14DV4 | TRAJ15 | CAMRPHQAGTALI | TRBV6-1 | TRBD2 | TRBJ1-2 | CAVGGGPGTNYGY |
| | | | F (SEQ ID NO. 275) | | | | TF (SEQ ID NO. 276) |
| CD4 TCR-6 | TRAV9-2 | TRAJ49 | CALSGWGSGNQF | TRBV7-3 | TRBD1 | TRBJ2-2 | CASSLTGTDNTGEL |
| | | | YF (SEQ ID NO. | | | | FF (SEQ ID NO. 278) |
| | | | 277) | | | | |
| CD4 TCR-7 | TRAV12-3 | TRAJ30 | CAMSAPYRDDKII | TRBV7-9 | TRBD1 | TRBJ2-7 | CASSSDMTESYEQ |
| | | | F (SEQ ID NO. 279) | | | | YF (SEQ ID NO. |
| | | | | | | | 280) |
| CD4 TCR-8 | TRAV13-2 | TRAJ8 | CAEKKGFQKLVF | TRBV4-1 | TRBD2 | TRBJ1-1 | CASSPGEGGGTEAF |
| | | | (SEQ ID NO. 281) | | | | F (SEQ ID NO. 282) |
| CD4 TCR-9 | TRAV29DV5 | TRAJ13 | CAASKVTF (SEQ | TRBV11- | TRBD1 | TRBJ1-6 | CASTPGTGYSPLHF |
| | | | ID NO. 283) | 2 | | | (SEQ ID NO. 284) |
| CD4 TCR-10 | TRAV38- | TRAJ42 | CAYRSPNYGGSQG | TRBV7-2 | TRBD1 | TRBJ2-7 | CASSLGRGGIYEQY |
| | 2DV8 | | NLIF (SEQ ID NO. | | | | F (SEQ ID NO. 286) |
| | | | 285) | | | | |
| | | | | b-V | b-D | b-J | |
| Tumor-3 | a-V gene | a-J gene | a-CDR3 | gene | gene | gene | b-CDR3 |
| CD8 TCR-1 | TRAV13-1 | TRAJ20 | CAAGDDYKLSF | TRBV20- | None | TRBJ1-1 | CSARDPRRTNTEAF |
| | | | (SEQ ID NO. 287) | 1 | | | F (SEQ ID NO. 288) |
| CD8 TCR-2 | TRAV25 | TRAJ38 | CAGASNAGNNRK | TRBV5-4 | TRBD2 | TRBJ2-3 | CASSLLLARRSDTQ |
| | | | LIW (SEQ ID NO. | | | | YF (SEQ ID NO. |
| | | | 289) | | | | 290) |
| CD8 TCR-3 | TRAV5 | TRAJ17 | CVLPPAAGNKLTF | TRBV2 | TRBD1 | TRBJ1-3 | CASSEQGSGNTIYF |
| | | | (SEQ ID NO. 291) | | | | (SEQ ID NO. 292) |
| CD8 TCR-4 | TRAV41 | TRAJ42 | CAVRRDGGSQGN | TRBV6-1 | TRBD2 | TRBJ1-4 | CASTPRGKGEKLFF |
| | | | LIF (SEQ ID NO. | | | | (SEQ ID NO. 294) |
| | | | 293) | | | | |
| CD8 TCR-5 | TRAV19 | TRAJ32 | CALGRRGGATNK | TRBV5-1 | TRBD2 | TRBJ2-5 | CASSTGLAGQETQ |
| | | | LIF (SEQ ID NO. | | | | YF (SEQ ID NO. |
| | | | 295) | | | | 296) |
| CD8 TCR-6 | TRAV36DV7 | TRAJ53 | CAVESGGSNYKLT | TRBV18 | TRBD1 | TRBJ1-1 | CASSLDWENTEAF |
| | | | F (SEQ ID NO. 297) | | | | F (SEQ ID NO. 298) |
| CD8 TCR-7 | TRAV38- | TRAJ41 | CARYSGYALNF | TRBV18 | TRBD2 | TRBJ2-1 | CASSPELADYNEQF |
| | 2DV8 | | (SEQ ID NO. 299) | | | | F (SEQ ID NO. 300) |
| CD8 TCR-8 | TRAV12-2 | TRAJ52 | CAVKSHRGTSYG | TRBV7-3 | None | TRBJ2-2 | CASSFGPGELFF |
| | | | KLTF (SEQ ID NO. | | | | (SEQ ID NO. 302) |
| | | | 301) | | | | |
| CD8 TCR-9 | TRAV13-1 | TRAJ23 | CAASPRIYNQGGK | TRBV19 | TRBD1 | TRBJ2-2 | CASSAVDRPTGELF |
| | | | LIF (SEQ ID NO. | | | | F (SEQ ID NO. 304) |
| | | | 303) | | | | |
| CD8 TCR-10 | TRAV20 | TRAJ33 | CAVRGMDSNYQLI | TRBV7-2 | TRBD1 | TRBJ2-7 | CASSLVGGSYEQY |
| | | | W (SEQ ID NO. 305) | | | | F (SEQ ID NO. 306) |

In order to verify whether these TCRs recognize tumor antigens, the B cells expressing the tumor antigens were mixed with each TCR-T cell line and cultured for antigen stimulation experiment, and the B cells not expressing tumor antigens were used as a control.

Firstly, the above B cells were mixed with the T cells at a ratio of 1:1, inoculated in a 96-well plate, and co-cultured for 48 h, and then, the IFNG level in the supernatant was detected by ELISA. If the tumor antigens expressed by the B cells could be recognized by the TCRs expressed by the T cells, then the T cells would be activated and secrete the effector cytokine IFNG. The more the T cells being activated, the more the generated IFNG. By comparing the level of IFNG in the supernatant, whether these TCR-T cells could be activated by the tumor antigens can be verified, so as to determine whether these TCRs can recognize the tumor antigens.

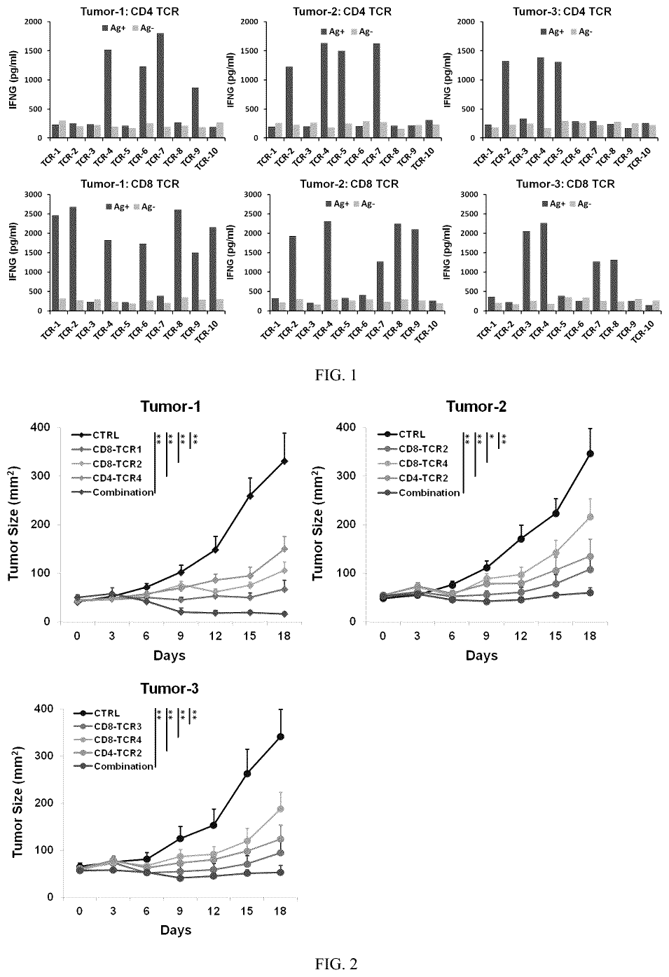

As shown in FIG. 1, the TCRs that recognized tumor antigens can be identified accurately using the established approach. Among the high-frequency TCRs of the first tumor tissue, four TCRs derived from CD4 T cell and seven TCRs derived from CD8 T cell can be activated by the B cells expressing the tumor antigens. The TCRs identified in the second tumor tissue that can recognize tumor antigens include 4 TCRs from CD4 T cells and 5 TCRs from CD8 T cells, respectively. In the third tumor tissue, there are 3 TCRs from CD4 T cells and 4 TCRs from CD8 T cells which can recognize tumor antigens. These results indicate that both T cells that can recognize tumor antigens and T cells that do not recognize tumor antigens are indeed present in the tumor.

Next, in order to verify whether these tumor-specific TCRs can be used for tumor therapy, the TCR-T cells that recognize tumor antigens were used to treat a tumor PDX model of the same origin. From the TCRs that have been verified to recognize tumor antigens, two TCRs with the highest frequency in CD8 T cells and one TCR with the highest frequency in CD4 T cells were selected and prepared for TCR-T cells for treatment. In addition, the three TCR-T cells were also mixed at a ratio of 1:1:1 for multi-TCR treatment. When the PDX tumors transplanted subcutaneously into NSG mice grew to about 50 mm² in size, 6×10⁶ TCR-T cells were injected into the mice for treatment through the tail vein.

As shown in FIG. 2, in the treatments for the three tumor PDXs, the treatment with the mixed three TCRs shows the best therapeutic effect, and the treatments with the single TCR also all show obvious therapeutic effects.

These results show that the tumor antigen-recognizing TCRs identified by the method according to the present disclosure not only recognize the tumor antigens, but also effectively treat the tumor, and the treatments with multiple TCRs simultaneously produces better therapeutic effect.

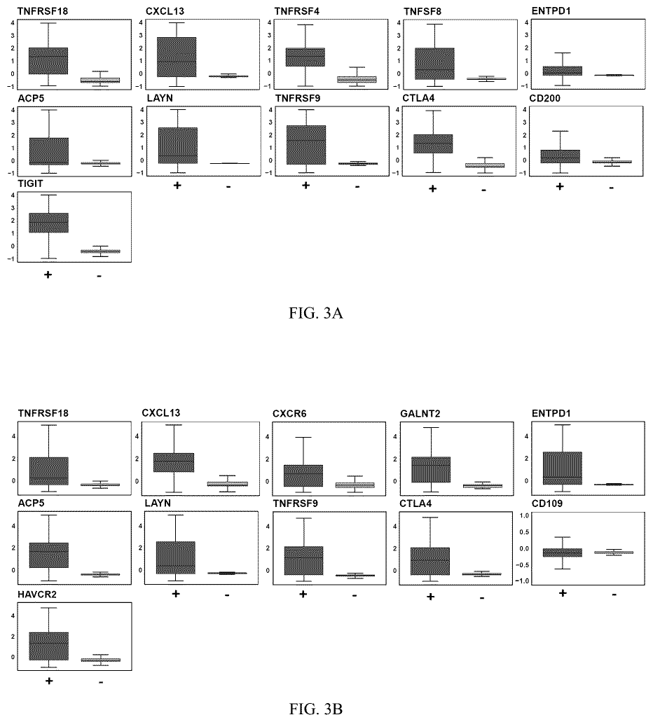

In order to identify the molecular markers of the subset of tumor antigen-specific T cells, CD4 and CD8 T cells carrying these TCRs were divided into two groups: tumor antigen-recognizing positive group and tumor antigen recognizing negative group, depending on whether the identified TCRs recognize tumors. Then, using the RNA sequencing results of each cell, the abundance of RNA expression of each gene was compared between the two groups of T cells to find out the differentially expressed genes. As a result, a number of genes specifically expressed in the T cells positive in tumor antigen recognition were found out. As shown, the tumor antigen-recognizing CD4 T cells specifically express: TNFRSF18, CXCL13, TNFRSF4, TNFSF8, ENTPD1, ACP5, LAYN, TNFRSF9, CTLA4, CD200 and TIGIT (FIG. 3A), and the tumor antigen-recognizing CD8 T cells specifically express: TNFRSF18, CXCL13, CXCR6, GALNT2, ENTPD1, ACP5, HAVCR2, LAYN, TNFRSF9, CTLA4 and CD109 (FIG. 3B). Among them, TNFRSF18, CXCL13, ENTPD1, ACP5, LAYN, TNFRSF9 and CTLA4 are highly expressed in both the tumor antigen-recognizing CD4 and CD8 T cells. By these specifically expressed genes, the T cells that recognize tumor antigens can be significantly distinguished from the T cells that do not recognize the tumor antigens. In addition, these specifically expressed genes can all be used to significantly distinguish the T cells that recognize tumor antigens from the T cells that do not recognize the tumor antigens in various tumor types, thus having a good broad spectrum. Therefore, these genes can be used as molecular markers to identify and isolate T cells that recognize tumor antigens from tumors and obtain the TCRs carried thereby for tumor treatment.

In order to further verify whether the T cells that recognize tumor antigens can be identified and isolated by these molecular markers, the molecular markers expressed on the surface of the T cells were selected as molecular markers for flow sorting, wherein TNFRSF18, TNFRSF4, ENTPD1, TNFRSF9, CTLA4, CD200 and TIGIT were selected for the tumor antigen-recognizing CD4 T cell, and TNFRSF18, CXCR6, ENTPD1, TNFRSF9, HAVCR2, CTLA4 and CD109 were selected for the tumor antigen-recognizing CD8 T cell. Firstly, according to the above-mentioned tumor tissue digestion method, five lung cancer tissues were digested into single-cell suspensions, and a portion of the single-cell suspension was centrifuged, resuspended in RPMI medium containing 10% FBS at a density of 1×10⁶ cells/ml, and then frozen to −80° C., as tumor antigens for the subsequent antigen stimulation experiment. The remaining single-cell suspension was divided into seven aliquots, which were respectively subjected to flow antibody stain. The antibody combinations were 1) CD4, CD8 and TNFRSF18; 2) CD4, CD8 and ENTPD1; 3) CD4, CD8 and TNFRSF9; 4) CD4, CD8 and CTLA4; 5) CD4, CD8, TNFRSF4 and CXCR6; 6) CD4, CD8, CD200 and HAVCR2; and 7) CD4, CD8, TIGIT and CD109. Then, the CD4 and CD8 T cells in the tumor tissue were sorted into two groups of molecular marker positive and negative cells respectively according to the staining of these molecular marker antibodies, and 14 groups of CD4 T cells and 14 groups of CD8 T cells were totally obtained.

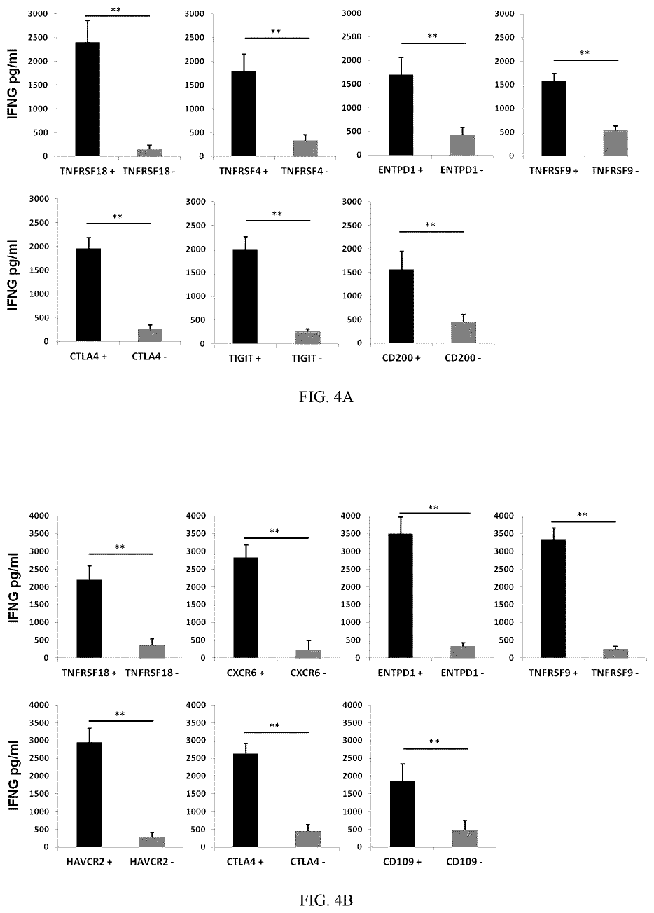

Each sorted T cells group was resuspended in RPMI medium containing 10% FBS and 200 ng/ml IL2 and inoculated into a U-shaped bottom 96-well plate coated with CD3/CD28 antibody for nonspecific activation. After one week of incubation, the T cells of each group were taken out. After washing with PBS twice, the T cells were resuspended in RPMI medium containing 10% FBS at a density of 2×10⁶/ml. 200 μL of cells were inoculated into a U-shaped bottom 96-well plate, and each group of cells were set in triplicate. In addition, the frozen single-cell suspension of tumor tissue was revived to 37° C. by a water bath, from which 50 μL was taken and added to the inoculated T cells as tumor antigen for specific activation. After activation for 20 hours, the content of IFNG in the culture medium was detected by ELISA to measure the response of the T cells in each group to the tumor antigen stimulation. The inventors found that among CD4 T cells, TNFRSF18+, TNFRSF4+, ENTPD1+, TNFRSF9+, CTLA4+, CD200+ and TIGIT+ T cells could all be activated by the tumor antigens, whereas the corresponding T cells negative in these molecular markers could not be activated (FIG. 4A). Similarly, among CD8 T cells, TNFRSF18+, CXCR6+, ENTPD1+, TNFRSF9+, HAVCR2+, CTLA4+ and CD109+ T cells could all be activated by the tumor antigens, whereas the corresponding T cells negative in these molecular markers could not be activated (FIG. 4B).

The above results indicate that these molecular markers can be used to isolate tumor antigen-recognizing T cells from tumors. The T cells separated and obtained by these molecular markers and the TCRs carried thereby can be used for the treatment of a tumor in a patient.

1. A TCR-T cell for tumor-killing, wherein the TCR-T cell is a T cell carrying a TCR that recognizes a tumor antigen, and the TCR in the TCR-T cell is derived from any one or more of the following T cells:
1) a CD4 T cell expressing one or more of TNFRSF18, CXCL13, TNFRSF4, TNFSF8, ENTPD1, ACP5, LAYN, TNFRSF9, CTLA4, CD200 and TIGIT genes in the tumor; and
2) a CD8 T cell expressing one or more of TNFRSF18, CXCL13, CXCR6, GALNT2, ENTPD1, ACP5, HAVCR2, LAYN, TNFRSF9, CTLA4 and CD109 genes in the tumor.

2. (canceled)

3. (canceled)

4. A method for preparing the TCR-T cell according to claim 1, comprising: identifying a TCR that recognizes a tumor antigen; and introducing one or more nucleotide sequences of the TCR that recognizes a tumor antigen into a T cell for expression to construct the TCR-T cell.

5. The method according to claim 4, wherein identifying a TCR that recognizes the tumor antigen comprises: by means of flow sorting, magnetic bead separation or tumor tissue in-situ sequencing, obtaining, from a tumor tissue, a T cell, which expresses any one or more of the markers of a CD4 T cell for tumor antigen recognition, selected from the group consisting of TNFRSF18, CXCL13, TNFRSF4, TNFSF8, ENTPD1, ACP5, LAYN, TNFRSF9, CTLA4, CD200 and TIGIT, and a TCR sequence carried thereby; and a T cell, which expresses any one or more of the markers of a CD8 T cell for tumor antigen recognition, selected from the group consisting of TNFRSF18, CXCL13, CXCR6, GALNT2, ENTPD1, ACP5, HAVCR2, LAYN, TNFRSF9, CTLA4 and CD109, and a TCR sequence carried thereby.

6. A method for identifying a T cell and a TCR for tumor antigen recognition, comprising: establishing a TCR-expressing TCR-T cell by cloning a high-frequency TCR in a tumor tissue, performing in vitro tumor antigen stimulation by using antigen-presenting cells expressing a tumor antigen tandem gene, and identifying a TCR carried by a TCR-T cell that can be stimulated as the TCR for tumor antigen recognition, and the T cell carrying such TCR as the T cell for tumor antigen recognition,
wherein the TCR in the TCR-T cell is derived from any one or more of the following T cells:
1) a CD4 T cell expressing one or more of TNFRSF18, CXCL13, TNFRSF4, TNFSF8, ENTPD1, ACP5, LAYN, TNFRSF9, CTLA4, CD200 and TIGIT genes in the tumor; and
2) a CD8 T cell expressing one or more of TNFRSF18, CXCL13, CXCR6, GALNT2, ENTPD1, ACP5, HAVCR2, LAYN, TNFRSF9, CTLA4 and CD109 genes in the tumor.

7. The TCR-T cell according to claim 1, wherein the TCR in the TCR-T cell is derived from any one or more of the following T cells:
1) a CD4 T cell expressing one or more of TNFRSF18, TNFRSF4, ENTPD1, TNFRSF9, CTLA4, CD200 and TIGIT genes in the tumor; and
2) a CD8 T cell expressing one or more of TNFRSF18, CXCR6, ENTPD1, HAVCR2, TNFRSF9, CTLA4 and CD109 genes in the tumor.

8. The method according to claim 5, wherein the one or more of the markers of a CD4 T cell for tumor antigen recognition are selected from the group consisting of TNFRSF18, TNFRSF4, ENTPD1, TNFRSF9, CTLA4, CD200 and TIGIT, and a TCR sequence carried thereby; and the one or more of the markers of a CD8 T cell for tumor antigen recognition are selected from the group consisting of TNFRSF18, CXCR6, ENTPD1, HAVCR2, TNFRSF9, CTLA4 and CD109, and a TCR sequence carried thereby.

9. The method according to claim 4, wherein the one or more nucleotide sequences of the TCR that recognizes a tumor antigen is introduced into a T cell by lentivirus infection.

10. The method according to claim 6, wherein the TCR in the TCR-T cell is derived from any one or more of the following T cells:
1) a CD4 T cell expressing one or more of TNFRSF18, TNFRSF4, ENTPD1, TNFRSF9, CTLA4, CD200 and TIGIT genes in the tumor; and
2) a CD8 T cell expressing one or more of TNFRSF18, CXCR6, ENTPD1, HAVCR2, TNFRSF9, CTLA4 and CD109 genes in the tumor.

11. A pharmaceutical composition, comprising the TCR-T cell according to claim 1.

12. A method for treating a tumor, comprising administering a therapeutic amount of the TCR-T cell according to claim 1 to a patient in need thereof.
